(19) United States
(12) Patent Application Publication
(10) Pub. No.: US 2021/0315492 A1
(43) Pub. Date: Oct. 14, 2021

(54) COMPOSITE MATRIX FOR ANALYTE BIOSENSORS

(71) Applicant: THE REGENTS OF THE UNIVERSITY OF CALIFORNIA, Oakland, CA (US)

(72) Inventors: Avid Najdahmadi, Irvine, CA (US); John Weidling, Irvine, CA (US); Elliot L. Botvinick, Irvine, CA (US)

(21) Appl. No.: 17/177,570

(22) Filed: Feb. 17, 2021

Related U.S. Application Data
(63) Continuation-in-part of application No. PCT/US19/46923, filed on Aug. 16, 2019.
(60) Provisional application No. 62/719,529, filed on Aug. 17, 2018.

(51) Int. Cl. A61B 5/1459; C08J 3/20; C08F 14/26; C09K 11/06; C08F 2/50; A61B 5/145; A61B 5/1473

(52) U.S. Cl. CPC A61B 5/1459; C08J 3/20; C08F 14/26; C09K 11/06; B82Y 5/00; A61B 5/14532; A61B 5/14503; A61B 5/14546; A61B 5/1473; C08F 2/50

(57) ABSTRACT

A composite material for detecting and measuring levels of analytes and target molecules in biological fluid and tissue is disclosed herein. This composite material is constructed from a porous mesh sheet that is coated with a signal emitting material and is impregnated with target detecting molecules mixed inside one or multiple polymer mixtures. The geometry and configuration of different constituents can provide high efficiency of signals, the ability to measure multiple signals, small dimensions, fast response to changes, long storage lifetimes, controlled diffusional and mechanical properties, as well as easy industrial manufacturability. The composite material can be used in many sensing applications including optical or electrochemical continuous biosensing such as continuous glucose and lactate monitors.

### CROSS-REFERENCES TO RELATED APPLICATIONS

This application is a continuation-in-part and claims benefit of International Application No. PCT/US2019/046923, filed Aug. 16, 2019, which claims benefit of U.S. Provisional Application No. 62/719,529, filed Aug. 17, 2018, the specification(s) of which is/are incorporated herein in their entirety by reference.

### GOVERNMENT SUPPORT

This invention was made with government support under Grant No. FA9550-17-1-0193 awarded by the United States Air Force Office of Scientific Research. The government has certain rights in the invention.

### FIELD OF THE INVENTION

Embodiments of the invention relate to analyte biosensors, in particular, to a composite material that can detect and measure levels of analyte(s) in biological fluids and tissues.

### BACKGROUND

Analyte biosensors are used to detect and measure the levels of analyte(s) in biological fluids and tissues. Examples of said biosensors include enzymatic biosensors, such as oxidase-based continuous monitoring biosensors, e.g., sensors that can measure analytes such as glucose and lactate. For diabetic patients, measurement of both of these analytes and their changes in tissue such as the subcutaneous space can result in a better understanding of the metabolic pathways, especially during and following periods of intense physical activity. The combined data can lead to a more accurate dosing of insulin and better glycemic control. Additionally, continuous measurement of lactate is a reliable indicator of shock in critical injury and diseases.

Existing biosensors that utilize enzymes whose reaction is monitored by luminescent dyes are typically constructed by having a dye volume juxtaposed to an enzyme volume. The enzyme and dye may be layered in a sandwich onto a platform responsible for excitation and/or detection of light (e.g., see FIG. 1C). In this embodiment, only the dye directly in contact with the enzyme emits light, which leads to reporting the analytes and/or products of the enzyme reaction. The remaining volume of the dye is typically insensitive to the reaction, but nonetheless emits light. As a consequence of this geometry, the addition of more dye does not increase the signal amplitude as related to analyte/product monitoring, but instead only contributes to an unwanted background.

Alternatively, the enzyme and dye may be incorporated into a hydrogel, which may be implemented within a tissue-integrating sensor. For example, a tissue-integrating sensor may feature one or more polymers, such as hydrogels, with dye particles and enzyme particles mixed together in the hydrogels. However, since dyes and enzymes are often not co-soluble in the water phase of the hydrogel, it is a significant challenge to uniformly distribute dye and enzyme within the gel. Moreover, hydrogels are characteristically soft and fragile, and thus must typically be formed on the sensing device itself, as opposed to being formed in a large-scale process and then subsequently being placed onto a sensing device. In fact, previous works have found the implementation of hydrogels was inhibitory for large-scale fabrication of such sensors. Further still, adhesion of such a hydrogel to a second surface presents challenges; in particular, hydrogels tend to swell or contract away from a surface onto which it was deposited during hydration and polymerization/crosslinking. Such second surfaces may have characteristic lengths of several to hundreds of microns, further inhibiting reliable placement of the hydrogels over thousands of units. Similar challenges exist for optical sensing, which is not dependent upon an enzyme reaction but is instead dependent on other factors, e.g., molecular probes such as FOrster Resonant Energy Transfer (FRET) probes, reversible binding probes, etc., that modify an optical signal in response to a changing concentration of an analyte.

### SUMMARY OF THE INVENTION

One embodiment of the present invention provides improved materials and strategies for co-immobilizing dyes and enzymes, assuring uniform distribution of the enzymes and dyes, allowing precise placement onto small second surface, and controlling material geometry in such a way that allows for manufacturing on a large scale. With respect to enzymatic applications, the present invention may provide for the reduction of active enzyme-dye media thickness (which may allow for the use of a reduced size insertion needle in some applications, thus reducing the pain of insertion); maintaining high activity and stability of the enzyme(s) overtime, both during operation and storage; improvement of mechanical and adhesion stability of the luminescence dye/enzyme component; enhancement of the luminescence dye-enzyme component attachment to a surface; and/or improved manufacturability.

The current invention provides a new composite material with the necessary components to detect and measure the level of analyte(s) in fluids. The composite material may be used in optical sensing devices as well as enzymatic sensing devices, and may also be used in other applications including, but not limited to, filtering of analytes and electrochemical sensing. For example, in the case of electrochemical sensing, the porous matrix could be metalized instead of dye coated. In another embodiment, the composite material may comprise a porous conductive material, or an electronically conductive hydrogel, or particles/additives that make the composite material electronically conductive.

In some aspects, the present invention features a novel composite material comprising: (1) a porous matrix (e.g., mesh), such as one comprising polytetrafluoroethylene (PTFE), which acts as a reinforcement constituent, (2) a luminescent substance, such as an oxygen-sensitive dye, formulated to coat at least part of the surface area of the porous matrix, and (3) a hydrogel filling said porous matrix, where the hydrogel may (but not necessary needs to) contain at least one additional substance, such as a catalyst (e.g., enzyme) or a binding protein. As used herein, a “porous matrix” includes a mesh of fibers or a solid scaffold through which pores pass, and may be used interchangeably with sheet or film. Further in this document, “mesh” can refer to either a mesh of fibers or a porous material.

As described above, there was a need to create a new manufacturing platform for biosensors. For this reason, the present invention developed a method where a porous matrix is coated with a dye and then the hydrogel/enzyme is embedded in the pores of dye-coated porous matrix to achieve the benefits of mixing the dye and enzyme without having to co-solubilize them, as well as providing strong mechanical and adhesion characteristics to the fabricated sensor film. This developed composite matrix not only provided all the targeted needs, but also provided very high signal levels and long stability and functionality.

In order to describe the utility of the invention, one can consider a non-limiting embodiment featuring an enzymatic analyte sensor, particularly one dependent upon enzymes of the class oxidoreductases. For instance, an enzyme-polymer mixture may fill the porous matrix. Examples of an oxidoreductase are oxidase-based enzymes, such as glucose oxidase, which catalyze a reaction that consumes both oxygen and the analyte, resulting in decreased oxygen levels. Oxygen changes may be recordable and compared to the surrounding matrix oxygen level, and therefore the analyte concentration can be calculated.

In this embodiment, the enzyme is contained with the hydrogel phase. The hydrogel phase of the composite material provides enzyme entrapment, immobilization, and stability. The mesh, also referred to as the reinforcement constituent, provides the hydrogel with protection from mechanical manipulations. The porous matrix (e.g., reinforcement constituent) is coated with an oxygen-sensitive luminescent dye such that the interpenetrating hydrogel is in contact with the dye-coated porous matrix. Without wishing to limit the present invention to any theory or mechanism, it is believed that this geometry affords several advantages: (1) The surface area of hydrogel-dye contact is very large as compared to a system utilizing a non-porous matrix (as shown in FIG. 1C), which is limited to a ‘sandwich’ geometry between the hydrogel phase and the dye-coated matrix. As a result, the present invention can achieve a given surface area of dye-hydrogel contact with an overall composite size at least one order of magnitude smaller, including thinner, than a non-porous matrix. Thus, for a given portion of material, the surface area to volume ratio is much higher for the composite than for the sandwich. (2) The composite is much stronger than a hydrogel alone, and thus for a thin geometry, the composite can be mechanically manipulated with forces consistent with manufacturing processes and use, whereas the hydrogel cannot. Because of this, composite material can be fabricated in large sheets or films (larger as compared to the size implemented in the sensors), cut into appropriate sizes for a sensor and then picked up from the sheet and deposited onto the sensor. (3) Even with the thin geometry, the diffusion characteristics of the composite can be altered by changes to the hydrogel phase. This is important in the case of the oxidase-based sensor, where the diffusion constant of the analyte within the hydrogel phase of the composite can be optimized for a desired measurable range of analyte concentration. (4) Additionally, the dimensions of the matrix, or reinforcement constituent, of the composite can be tuned to optimize factors such as mechanical strength, total dye mass (and thus brightness), analyte diffusion path lengths, and thus response time to changes in bulk analyte concentration. (5) Further, due to the invented geometry and engineered constituent configurations, the thin composite matrix provides fast dynamics without necessitating tissue and blood vessel infiltration. In fact, the composite matrix was designed with characteristic pore sizes on the order of several microns or smaller to prevent cells from infiltrating the matrix, hence preventing tissue in-growth, and thus reducing local tissue tearing at the implantation site when the sheet is removed from the tissue.

In some aspects, the present invention features a method of producing the composite material, e.g., for improving continuous measurement of analytes in oxidase-based photonic biosensors or other appropriate application. This may be achieved by: (1) minimizing the thickness of the analyte detecting component down to 10 μm, or perhaps thinner; (2) increasing the stability of the enzyme components; (3) maintaining the enzyme activity thorough entrapment immobilization; (4) providing high mechanical stability; (5) limiting diffusion rate through the composite matrix structure and increasing the analyte sensitivity range; and (6) providing high luminescence signal for detection.

One of the unique and inventive technical features of the present invention is the entrapment of the enzyme or target-detecting molecule in a hydrogel matrix and the hydrogel matrix in a porous material, thereby allowing for mechanical manipulation of the hydrogel-containing composite sheet, where otherwise, the hydrogel on its own is easily damaged by mechanical manipulations. In one embodiment, that material comprises porous PTFE, a known hydrophobic compound and the hydrogel is hydrophilic. One of ordinary skill in the art would expect a hydrophilic hydrogel to be repelled out of the hydrophobic PTFE sheet, therefore it is counterintuitive to add the hydrophilic hydrogel to the hydrophobic PTFE sheet. However, the inventors have developed a method of producing the composite matrix capable of retaining the hydrophilic hydrogel containing the enzyme or target-detecting molecule within the hydrophobic sheet. Contrary to previous teachings, the present invention provides a composite matrix comprising a hydrophilic hydrogel entrapped in a hydrophobic sheet.

Without wishing to limit the present invention to any theory or mechanism, it is believed that one vital aspect of the present technology is that the porous mesh has dye coating, which is an important property. This configuration allows for an increased light output by adding more dye, wherein the dye stays near to the enzymes. As previously discussed with respect to the traditionally-used sandwich-style sensor configuration shown in FIG. 1C where a layer of enzyme is stacked on top of a layer of dye, the only way to make it brighter is to increase the thickness of the dye slab. However, incorporating an enlarged dye slab would not work because most of the dye is far from the reaction and thus would only provide a background signal that is independent from what is being measured.

As previously discussed, the present invention allows for smaller sensor dimensions and therefore less painful subcutaneous insertion, better mechanical stability and attachment due to use of the reinforcing constituent or mesh (e.g., PTFE or other appropriate mesh, biocompatible adhesives, etc.), and lower effective diffusion rates through the composite due to the porous mesh (e.g., PTFE). For example, the sensor has a thickness ranging from about 10 μm to about 100 μm, however, based on the final application, the sensors can also be made to have thicknesses differing from this range by orders of magnitude (thinner or thicker). Possessing a torturous path allows for longer diffusion paths without increasing bulk dimensions, and the hydrogel constituent has a tunable pore size characteristic that can determine its diffusion constant. These characteristics can increase analyte sensitivity range with faster dynamics (and one could excite at different points) and result in more secured protection for the reagent and as lowering the possibility of leaching in the body.

Additional features may include higher activity through physical entrapment of enzyme in the chains of the crosslinked hydrogel, higher emission of the dye and increased signal due to higher capacity for accommodating more dye in the porous PTFE as compared to other dye coating methods, increased storage and operational ability of the sensor because of the fabrication process, and repeatability and ease of fabrication that can lead to lower manufacturing costs. None of the presently known prior references or work has the unique inventive technical features of the present invention.

In some embodiments, the present invention also provides a composite matrix featuring a non-enzymatic target-detecting system. In one embodiment, the composite matrix may comprise a porous mesh material, and a target-detecting polymer mixture entrapped in the porous mesh material. The porous mesh material may comprise a polymeric network of fibers and pores or gaps between the fibers. The at least one target-detecting polymer mixture may fill at least a portion of the pores or gaps between the fibers. For example, the target-detecting polymer mixture can interpenetrate or fill the pores of the porous mesh material. In some embodiments, the composite matrix is configured to measure levels of a target. In one such embodiment the target-detecting polymer mixture would not necessarily contain enzymes, but may also contain at least a reversible binding molecule(s) that modulate a signal in response to binding a target molecule such as a hormone or peptide (e.g., insulin, glucagon) or a metabolite.

In other embodiments, the composite matrix may further comprise a luminescent material coating at least a portion of the porous mesh material. For example, the luminescent material may coat at least a portion of polymer fibers or pore walls that make up the porous mesh material. The luminescent material may also be disposed between the polymer fibers or pore walls, or entrapped between the polymer fibers of the porous mesh. In one embodiment, the luminescent material is a dye. In some embodiments, the target-detecting molecules may interact with the target in such a way as to absorb or bind to or release or produce or consume a substance after interaction with a target. The substance or target can alter one or more luminescent properties of the luminescent material.

In some embodiments, the at least one target-detecting polymer mixture may comprise a polymer and target-detecting molecules. In one embodiment, the target-detecting molecules are non-covalently entrapped within the polymer or covalently bound to the polymer. Alternatively or in conjunction, the polymer can be polymerized to entrap the target-detecting molecules in the polymer. In this case, “entrapped” refers to entrapment or localization of target-detecting molecules, such as enzymes, within a polymeric network in such a way that allows the substrate and products to pass through but retains the target-detecting molecule within the polymer.

In a non-limiting embodiment, the target-detecting molecule mixture comprises a hydrogel and target detecting molecules entrapped in the hydrogel. For example, the target detecting molecules may be non-covalently entrapped within the hydrogel, or covalently bound to the hydrogel, etc. In some embodiments, the hydrogel can be polymerized to entrap the target detecting molecules.

In some embodiments, the at least one target-detecting polymer mixture may comprise two or more polymer mixtures. Each polymer mixture of the two or more polymer mixtures may form a separate phase within the porous mesh material. In some other embodiments, the at least one target-detecting polymer mixture may comprise at least two different types of polymers having similar or different molecular weight cutoffs.

In some embodiments, molecular weight cutoffs can range from about 5 to 20 kDa for small peptides, and about 100 to 150 kDa for antibodies. In a non-limiting example, two different types of polymers having molecular weight cutoffs within 1 kDa are considered similar, and anything greater are considered different. In other embodiments, two different types of polymers having molecular weight cutoffs within 1-2 kDa are considered similar, and anything greater are considered different.

Without wishing to be bound to a particular theory or mechanism, the top polymer layer can act as a filter of unnecessary proteins or enzymes or drug molecules, which can interfere with the function or affect the binding proteins within the matrix, by providing a molecular weight cut off filtration, diffusion controlling method as well as protective membrane to entrap the targeting molecule in the bottom polymer layer.

In a non-limiting embodiment, the composite matrix comprises a porous mesh sheet, a dye (wherein the dye coats at least a portion of the porous mesh sheet or is entrapped between the fibers of the porous mesh sheet), and one or multiple layers of hydrogel entrapped in the porous mesh sheet. Each hydrogel layer may have a different molecular weight than the other layer. For example, two different types of hydrogels with different molecular weights are polymerized on opposite sides of the porous mesh.

In other embodiments, the at least one target-detecting polymer mixture may further comprise particles or nanoparticles, cofactors, enzymes, or combinations thereof. In a non-limiting embodiment, the target-detecting polymer mixture is a protein-polymer mixture that includes at least one enzyme. The enzyme molecules of the enzyme-polymer mixture may be non-covalently entrapped within or covalently bound to the polymer.

Non-limiting examples of targets include small molecules, ions, lipids, amino acids, peptides, polypeptides, proteins, glycoproteins, saccharides, nucleic acids, nucleic acids fragments, insulin, glucose, lactate, oxygen, glucagon, cholesterols, triglycerides, HDL, glutamine, lactose, sucrose, pyruvate, cytokines, chemokines, eicosanoids, glycated hemoglobin (A1C), leptins, troponin, drug molecules, myoglobin, dopamine, serotonin, sodium, magnesium, calcium, potassium ions, hormones, metabolites, glycerol, ammonia, ketone, cortisol, ethanol, methanol, CO₂, pH, temperature, or a combination thereof. The targets may be those that are important in health care and prevention. In addition to monitoring of analytes that are produced inside the body, the composite sheet can be used to detect and monitor levels of exogenous molecules that is of important for patients, healthcare professional, and hospitals. Examples of such monitoring can include, but is not limited to, continuous monitoring of food additives, caffeine, alcohol, nicotine, lead, vitamins, and other substances. Another example is continuous measurement of chemotherapeutic agents for cancer treatment, which can provide feedback on the drug concentration in the patient's body and guide treatment.

According to some embodiments, the present invention also provides sensors. In one aspect, the sensor comprises a light source, and the composite matrix is disposed within the light path of the light source. In some embodiments, the sensor further comprises a detector circuit capable of recording light emission from the dye coating the mesh sheet placed within the path of the emission light. In some embodiments, the light source comprises a light-emitting diode (LED). In some embodiments, the composite matrix is attached to the LED via attachment to an adhesive. For example, the hydrogel-infused, dye-coated composite mesh is adhered to LEDs mounted on flexible circuits to form a sensor that is inserted underneath the skin of a subject. The LEDs excite the dye and a photodetector placed in the path of emission above the skin detects the intensity and dynamics of the emission which corresponds to the level of the analyte. In another embodiment, the detector may be implanted with the sensor instead of placed above the skin. In an alternative embodiment, the composite matrix may be implanted while the light source and detector are exterior to the skin, e.g. the composite matrix is subcutaneous and the light source and detector are above the skin.

In other embodiments, the composite matrix is conductive and capable of carrying electrons. In one embodiment, the target-detecting polymer mixture may be electrically conductive. For instance, the one or more layers of hydrogel may be conductive for use with electrochemical sensing techniques. The conductive composite matrix may be adhered to an electrode and placed under the skin for use in electrochemical detection of analytes. In one embodiment, a detector may be placed exterior to the skin, for example, above the skin. In another embodiment, the detector may be implanted with the matrix and electrode.

In some embodiment, the sensor comprises a double layer composite that contains two different layers of polymer mixtures, each containing different target detecting molecules to measure different analytes. In this embodiment one layer can have conductive characteristics, while the adjacent layer possesses luminescent properties. Such composite material can be used in electrode based electrochemical sensing of analytes as well as optical sensing, or it can be used for both methods simultaneously. In these embodiments, the composite is adhered to an electrode from the conductive end and inserted inside the body, with the luminescent layer facing the skin, while a light source and detector are outside and placed above the skin. In this example, two different reactions with two different analytes can occur within one composite film in which one set of reaction results in electron release and the second does not, and the second only affects other properties of the composite material (spectral characteristics, volume change, etc.). Both reactions can be recorded through simultaneous electrochemical and optical sensing.

In one embodiment, the composite matrix may be used to create biosensors that can continuously measure one or multiple analytes. In such applications, the analyte sensing system is comprised of the following non-limiting examples: the composite matrix, LEDs and electrodes, flexible circuits, signal detector, signal transmitter and receiver, signal computing processor, battery, data storage component, and data transmitter. Such system can process the data or transmit the data to systems such as hand-held devices, smart phone, smart watch and computers. The data collected from such sensors can be analyzed using a mathematical algorithm to predict the upcoming changes in levels of analytes or correlated hormones. In some embodiments, the continuous biosensor system can work in conjunction with secondary systems to deliver therapeutics based on the measured levels of analytes. For example, such secondary systems can be an insulin pump system to create a closed loop system for advanced glycemic control, working in conjunction with the analyte sensor having the composite matrix.

According to other aspects, the sensors described herein may be utilized in methods for monitoring levels of an analyte in a subject. In some embodiments, the method comprises implanting into the subject a sensor according to the present invention, exciting the dye in the composite matrix using the light source, thereby causing the dye to emit light, and detecting an intensity and dynamics of the emitted light, wherein the intensity or dynamics of emitted light corresponds to the levels of the analyte. In one embodiment, the light source is attached to the composite matrix. Alternatively, the light source may be the light going through the skin of the subject. The light source excites the dye in the composite matrix, thereby causing the dye to emit light. The present invention is not limited to implantation in the dermis and subcutaneous space. In other embodiments, the composite matrix may be inserted or implanted anywhere in the subject, including, but are not limited to, peritoneal cavity, muscle tissue, visceral fat, gums, cheek, eye, ear, nose and etc.

In some embodiments, the biosensing technology of the present invention may be used in animal and clinical research, personal health monitoring, physician monitoring of patients, urgent care facilities, hospitals, emergency rooms, ICUs and other emergency departments. For example, lactate trends can be used to improve treatment in life threatening situations. Continuous lactate biosensors can be used in critical care for lactate guided therapy as its level rises during critical illness conditions. Non-limiting examples of conditions in which the continuous analyte biosensors may be used are diabetes care and health monitoring, liver diseases, Parkinson's disease, trauma, sepsis, hemorrhagic shock, pulmonary embolism, cardiogenic shock, aortic aneurysm, carotid artery disease, hyponatremia, heart failure, sudden cardiac arrest, acute respiratory distress syndrome, acute respiratory failure and pneumonia, idiopathic pulmonary fibrosis, heart diseases and strokes, congestive heart failure, acute decompensated heart failure, peripheral vascular resistance, fertility status, inflammatory responses, dehydration, hemodialysis, stress assessment, metabolism status, lifestyle and fitness monitoring, cancer detection, therapeutic drug monitoring, including drug concentrations or drug response indicators, infectious disease monitoring, pesticide monitoring, heavy metal monitoring, food additive monitoring, and the like.

In some embodiments, the present invention may be used for drug monitoring in which the level of drug molecules within the interstitial fluid is detected. The drug levels may be detected periodically or continuously. In other embodiments, the present invention may be used for monitoring tissue hydration. The sensor may be used to measure tissue water content by determining the change in water content in the hydrogel. The present invention may be utilized in sports medicine for monitoring total body hydration such as estimating water loss in muscles, as well as medical applications such as evaluating increased fluids content in the tissue caused by the variety of pathological conditions or edemas.

According to other aspects, the present invention also provides methods for producing a composite matrix. One such method may comprise infusing a porous mesh material with one or more target-detecting polymer precursor mixtures so as to fill the pores of the porous mesh material with the one or more target-detecting polymer precursor mixtures in the porous mesh material, and curing the one or more target-detecting polymer precursor mixtures, thereby forming the composite matrix. In a further embodiment, a luminescent material may be introduced into the porous mesh material prior to infusion such that the luminescent material coats at least a portion of the porous mesh material.

Another method of producing a composite matrix may comprise the steps of introducing a luminescent material into a porous mesh material, where the luminescent material coats at least a portion of the porous mesh material, infusing the porous mesh material with one or more target-detecting polymer precursor mixtures, where the one or more target-detecting polymer precursor mixtures becomes entrapped in the porous mesh material, and curing the one or more target-detecting polymer precursor mixtures, thereby producing the composite matrix.

In some embodiments, the method may further include compressing or applying pressure to the porous mesh material along with the luminescent material and/or the one or more target-detecting polymer precursor mixtures, thereby increasing penetration of the luminescent material and/or the one or more target-detecting polymer precursor mixtures into the porous mesh material. In yet other embodiments, the method may further include plasma-treating the porous mesh material with the luminescent material, thereby increasing hydrophilicity of the porous mesh material with the luminescent material.

In another non-limiting embodiment, the method may comprise introducing a dye to a porous mesh sheet, wherein the dye coats at least a portion of the porous mesh sheet; infusing the porous mesh sheet with an enzyme-polymer mixture, wherein the enzyme-polymer mixture becomes entrapped in the porous mesh sheet; and curing the enzyme-polymer mixture, thereby producing a dye-coated, enzyme-filled composite matrix. In some embodiments, the method further comprises plasma-treating a surface of the porous mesh sheet with dye, thereby increasing hydrophilicity of the porous mesh sheet with dye. In some embodiments, the method further comprises compressing the porous mesh sheet with dye and enzyme-polymer mixture, thereby helping the enzyme-polymer mixture to penetrate the porous mesh sheet and limit a final thickness of the composite matrix.

According to other aspects, the present invention also provides methods for fabricating a sensor. One fabrication method may comprise coating at least a portion of a porous mesh material with a luminescent material, infusing the porous mesh material with one or more target-detecting polymer precursor mixtures, where the one or more target-detecting polymer precursor mixtures becomes entrapped in the porous mesh material, curing the one or more target-detecting polymer precursor mixtures to produce a composite matrix, and attaching the composite matrix to a light source. In some embodiments, the composite matrix is attached to the light source via an adhesive. In some embodiments, the method further comprises plasma-treating a surface of the porous mesh material with the luminescent material, thereby increasing hydrophilicity of the porous mesh sheet with the luminescent material. In some embodiments, the method further comprises comprising compressing the porous mesh sheet with the luminescent material and target-detecting polymer precursor mixtures, thereby helping the target-detecting polymer precursor mixtures to penetrate the porous mesh material and limit a final thickness of the composite matrix.

In another embodiment, the method of fabricating a sensor may comprise coating a mesh sheet with a dye; infusing the mesh sheet with an enzyme mixture; curing the enzyme mixture, thereby producing a dye-coated, enzyme-filled composite matrix; and attaching the composite matrix to a light source. In some embodiments, the composite matrix is attached to the light source via an adhesive. In some embodiments, the method further comprises plasma-treating a surface of the porous mesh sheet with dye, thereby increasing hydrophilicity of the porous mesh sheet with dye. In some embodiments, the method further comprises comprising compressing the porous mesh sheet with dye and enzyme-polymer mixture, thereby helping the enzyme-polymer mixture to penetrate the porous mesh sheet and limit a final thickness of the composite matrix.

With respect to any of the aforementioned compositions or methods, in some embodiments, the mesh sheet comprises a polymeric network of fibers and pores or gaps between the fibers. In some embodiments, the luminescent material is a dye. The dye coats at least a portion of the polymer fibers. In some embodiments, the dye is entrapped between the polymer fibers. In some embodiments, the enzyme-polymer mixture fills at least a portion of the pores or gaps between the fibers.

In some embodiments, the porous mesh sheet is a polytetrafluoroethylene (PTFE) sheet or an expanded polytetrafluoroethylene (ePTFE) sheet. In some embodiments, the porous mesh sheet comprises PTFE, ePTFE, alumina oxide, cellulose acetate, ceramics, sol gels, glass fiber filets, mixed cellulose ester, nylon, ethylene tetrafluoroethylene (ETFE), fluorinated ethylenepropylene (FEP), perfluoro-alkoxy (PFA), polyacrylonitrile (PAN), POTS (polycarbonate), PEEK (polyether ether ketone), polyethersulfone (PES), polyester, polypropylene, polyvinylidene fluoride (PVDF), or a combination thereof. In some embodiments, the porous mesh sheet comprises a ceramic or a metal. In some embodiments, the metal is gold, silver, aluminum, platinum, tungsten, stainless steel, nitinol, copper, niobium, titanium, or a combination thereof.

In some embodiments, the porous mesh sheet may be fabricated from material made porous through methods such as 3D printing, laser cutting, salt leaching, connecting fibers, etching, etc. Such materials can be made of synthetic polymers, naturally occurring polymers, or combinations thereof. Non-limiting examples of such polymers include: 2-hydroxy methacrylate (HEMA), polyethylene glycol (PEG), polyvinyl alcohol, polyacrylamide and their mixtures, silicone, polyurethane, alginate, complex carbohydrates, extracellular matrix, fibronectin, chitosans, albumin, collagens, etc. In other embodiments, the porous mesh sheet may be fabricated from non-polymer materials such as ceramics or metals.

In some embodiments, the dye comprises a polymer. In some embodiments, the polymer of the dye comprises polystyrene, poly (styrene-co-acrylonitrile), fluoroacrylic sol-gel poly(norobornene)s, polyvinylchloride, hydrophilic poly(2-hydroxyethyl methacrylate)-co-polyacrylamide, hydrophobic polystyrene, silicon rubbers, organically modified silica, ethyl cellulose, PVC, polysulfones, poly (dimethylsiloxane), or a combination thereof. In some embodiments, the polymer entraps the dye. In some embodiments, the dye is a luminescent dye. In some embodiments, the luminescent dye is an oxygen sensitive dye, a pH sensitive dye, a carbon dioxide sensitive dye, a sodium sensitive dye, a potassium sensitive dye, a magnesium sensitive dye, a thiol sensitive dye, or a calcium sensitive dye. Examples of ions sensitive dyes include, but are not limited to, SBFI, PBFI, FIuxOR™ dye, Fluo-4, Fluo-5F, Alexa Fluor™, Magnesium Green, mag-fluo-4, and Oregon Green™. In some embodiments, the dye is Pt(II) meso tetraphenyl tetrabenzoporphoryn (PtTPTBP), PtOEP, PdOEP, PtTFPP, PdTFPP, PtOEPK, PdOEPK, PtIFPPL, PdTFPPL , PdTPTBP, PtTPTBPF, PdTPTBPF, Pt1NF, Pd1NF, Pt2NF, Pd2NF, Pt3NF, Pd3NF, PtTPTNP, PdTPTNP, PtTBP(CO₂Bu)₈, PdTBP(CO₂Bu)₈, PtNTBP, PdNTBP, Oxyphor R2, Oxyphor G2, PtTCPP, Ir(III), Pt(II), Ir(ppy)₃, Ir(ppy-NPh₂)₃, Ir(btpy)₃, [Ru(bpy)₃]²⁺, or [Ru(dpp)₃]²⁺.

In some embodiments, the enzyme-polymer mixture comprises a hydrogel and enzyme molecules entrapped in the hydrogel. In some embodiments, the hydrogel is polymerized to entrap the enzyme molecules in hydrogel. In some embodiments, the hydrogel is photocured using a photoinitiator. In some embodiments, the enzyme molecules are non-covalently entrapped within the hydrogel. In some embodiments, the enzyme molecules are covalently bound to the hydrogel. In some embodiments, the hydrogel comprises polyethylene glycol (PEG), poly(ethylene oxide), poly(3,4-ethylenedioxythiophene), bis-poly(ethyleneglycol) lauryl terminated, polyethylene glycol monodisperse solution, 4arm-PEG, catalase-polyethylene glycol, O,O'-Bis[2-(N-Succinimidyl-succinylamino)ethyl] polyethylene glycol, poly(ethylene glycol) methyl ether methacrylate, diethylene glycol butyl ether methacrylate, poly(ethylene glycol) methacrylate, poly [silicone-aft-PEG] dimethacrylate, poly(ethylene glycol) dimethacrylate, tetraethylene glycol dimethacrylate, tetra(ethylene glycol) diacrylate, Tri(ethyleneglycol) diacrylate, poly(ethylene glycol) diacrylate, PEG-polyisobutylene, PEG-poly(c-benzyl L-glutamate), polyacrylamide (PAAm), poly(N-isopropylacrylamide) (PNIPAAm), PEG-PNIPAAm, collagen, fibrin, alginate, agarose, dextran, poly(hydroxyethyl methacrylate) (PHEMA), poly(vinylpyrrolidone) (PVP), pullulan, poly-lactic-co-glycolic acid (PLGA), poly(butylene terephthalate), poly(2-ethyl-2-oxazoline)-PCL, PLLA, PDLA, poly(vinyl alcohol) (PVA), chitosan-PVA, gelatin, albumin, polysaccharides, polyesters, polyamides, poly(oligoethylene glycol methacrylate) (POEGMA), poly(vinylpyrrolidinone), phosphorylcholine, poly(-oxazoline), polyethylenimine (PEI), poly(acrylic acid), polymethacrylate, polyelectrolytes, hyaluronic acid, chitosan-based hydrogels, chondroitin sulfate, methylcellulose, hydroxypropylcellulose, hydroxypropylmethylcellulose, amylose, amylopectin, carrageenan, hydroxyethyl methacrylate (HEMA), 2-HEMA, poly(2-HEMA), poly-L-lysine, poly(propylene fumarate) cellulose derivatives, elastin, calcium polyphosphate, or a combination thereof.

In further embodiments, additives may be incorporated in the hydrogel matrix. In some embodiments, these additives are cofactors that work in parallel with target detection molecules. Alternatively, or in conjunction, target detecting molecules can be labeled with an additional molecule. In some embodiments, these additives are particles/nanoparticles. In one embodiment, the particles are placed inside a stabilizing gel and mixed into the hydrogel matrix. In another embodiment, the particles may be covalently or non-covalently immobilized into the hydrogel matrix. Without wishing to limit the present invention, incorporation of the particles can result in enhanced mechanical and spectral properties of the composite as well as magnetic characteristics or electrical conductivity of the composite film, which can be utilized for electrode-based electrochemical analyte sensing as well as optical analyte sensing. The composite matrix with additional additives can be used in sensing different stimuli though recording different stable signals that may include, but are not limited to, optical, electrochemical, electrical, temperature, pressure, ultrasonic, acoustic, and radiation signals.

In one embodiment, additives can be used to detect volume changes in the hydrogel matrix. In such cases, a volume change of the hydrogels (swelling/shrinking) can result in changes in electrical or optical response of the composite sheet, and such changes compared to a reference can correlate to changes in hydration of the subject. In another embodiment, the composite matrix with or without additives may be used in point-of-care diagnostics to measure levels of analytes in small blood drops.

In some embodiments, these particles can be metal based, ceramic based, or polymer based. Examples of additive particles include but are not limited to: gold nanoparticles, silver nanoparticles, platinum nanoparticles, iron oxide, zinc oxide, titanium dioxide, cadmium selenide (CdSe) or cadmium telluride (CdTe). In other embodiments, the additives may include, but are not limited to, carbon nanotubes, quantum dots, and additional target detecting molecules including FRET molecules such as luminescent lanthanide cryptates. For example, changes in volume within the hydrogel infused composite matrix that includes gold nanoparticles as additives can result in changes in optical diffraction of the matrix, and therefore a change in a detectable signal that can correlate to hydration. As another example, the composite matrix can include a separate pH sensitive luminescence molecule as an additive, which can report pH changes within the matrix caused by changes in emission peak of a luminance which is chosen to be spectrally distinct from the analyte sensing or oxygen sensing dye component. In such embodiments, the sensing characteristics of the composite matrix can be altered or advanced to generate signals that can detect different and additional analytes or stimuli. Such additives can result in detectable signals that would indicate changes in response to a stimulus. Changes within the matrix that can be recordable through introducing such additives are for example changes in volume (swelling/shrinking), pressure, temperature, optical diffraction, absorbance FRET or quenching, as well as other measurable responses to a stimulus.

In some embodiments, the composite matrix is configured to detect and measure levels of a target. In some embodiments, the composite matrix is configured to detect and measure analyte levels from an enzyme reaction. In some embodiments, the enzyme molecules are glucose oxidase or lactate oxidase. In some embodiments, the analyte is consumed or produced from an oxidase enzyme reaction.

In other embodiments, the enzymes may belong to the family of oxidoreductases. In this case, the matrix may also contain cofactors including but not limited to NADP and NAD+. In this embodiment, the electron transfer within a conductive hydrogel matrix can be recorded and correlated to the level of analytes in case of electrochemical sensors. In case of optical sensors, the changes in H+ due to the biochemical reactions can affect the emission dynamic of pH sensitive embedded luminescence dye. Examples of such enzyme molecules include but are not limited to: alcohol dehydrogenase, glycerol dehydrogenase, galacticol 2-dehydrogenase, aldehyde reductase, L-lactate dehydrogenase, D-lactate dehydrogenase, glycerate dehydrogenase, 3-hydroxygutanoate dehydrogenase, 3-hydroxyisobutyrate dehydrogenase, glucose 1-dehydrogenase, D-galactose 1-dehydrogenase, 3-hydroxypropionate dehydrogenase, and the like.

Any feature or combination of features described herein are included within the scope of the present invention provided that the features included in any such combination are not mutually inconsistent as will be apparent from the context, this specification, and the knowledge of one of ordinary skill in the art. Additional advantages and aspects of the present invention are apparent in the following detailed description and claims.

### BRIEF DESCRIPTION OF THE DRAWINGS

The features and advantages of the present invention will become apparent from a consideration of the following detailed description presented in connection with the accompanying drawings in which:

FIG. 1A shows an example of a composite of present invention comprising porous dye coated mesh containing enzyme mixture. This also illustrates the composite geometry and the entrapment of the enzyme mixture inside the dye-coated porous mesh.

FIG. 1B shows an example of a composite comprising porous dye coated mesh containing two layers of enzyme mixtures.

FIG. 1C (prior art) shows a sandwich layer design with block on dye (bottom layer of schematic) and enzyme (top layer of schematic).

FIG. 2 shows a non-limiting example of a method for preparing the composite matrix (colors shown are for illustrative purposes only and do not necessarily reflect the colors of the dye, etc.). A dye containing solution was added to both sides of a porous mesh (e.g., PTFE) and left overnight to evaporate solvents such as chloroform from the porous mesh (e.g., PTFE). The dye-sheet was then plasma treated on both sides: the sheet was taken out after 5 minutes of plasma treating, flipped, and then plasma treated for another 5 minutes with the other surface facing upward. Next, enzyme mixture was added to both sides of the dye-sheet. The enzyme mixture-dye-sheet was placed between substrates (e.g., glass substrates) and pressure was applied to both substrates. The enzyme mixture-dye sheet was then exposed to UV light for UV polymerization. The polymerized enzyme mixture-dye sheet was detached from the substrates.

FIG. 3 shows a non-limiting example of preparing a biosensor using a composite matrix of the present invention. Sheets (with and without enzyme hydrogel) were cut to small pieces. Small LEDs were soldered onto a flex circuit as the light source. The LEDs surfaces were knife coated with adhesive and the cut sheets were pressed to adhere. The flex circuit was kept in a dark environment for 24 hours prior to first use to allow the adhesives to fully cure.

FIGS. 4A-4B show activity test results of 10 μm porous PTFE-PEGDM sensors for Day 2 (FIG. 4A) and Day 10 (FIG. 4B). LED1 comprised a composite matrix with an enzyme mixture in PEGDM. LED2 is a reference sensor.

FIGS. 5A-5B show activity test results of a hydrophilic porous ePTFE-PEGDM (Poly (ethylene glycol) dimethacrylate) sensor (FIG. 5A) and 60 μm porous PTFE-PEGDM sensor (Day 11) (FIG. 5B).

FIG. 6A shows calibration results for an enclosed chamber with 16 composite sheets arranged in a 4×4 array (2-month Experiment-Dry Storage). The photograph shows a fully sealed chamber. Fluids injected are 1× PBS, 1 mM, 4 mM, 10 mM, 15 mM and 20 mM glucose. 5% gas is injected into the chamber with a long needle. There is one fluid exchange outlet and one gas outlets. PEGDM was used as the hydrogel in the enzyme mixture. The dots represent the indexes of fluid exchange found by MATLAB. Row 1 (LEDs 1-4, porous ePTFE hydrophilic+dye) and Row 2 (LEDs 5-8, porous PTFE 60 μm+dye+Enzyme mixture) were oxygen sensors as reference, and Row 3 (LEDs 9-12, porous PTFE 60 μm+dye+Enzyme mixture) and Row 4 (LEDs 13-16, porous ePTFE hydrophilic+dye+Enzyme mixture) were glucose sensors.

FIG. 6B shows calibration results for an enclosed chamber with 16 composite sheets arranged in a 4×4 array (2-month Experiment-Dry Storage) as described in FIG. 6A. The calibration results from 6 inserts were graphed and analyzed, a polynomial fit was implemented for the glucose sensors results (LEDs 9, 11, 12).

FIGS. 7A-7C show sensor calibration after 5 months of storage.

FIGS. 7D-7F show sensor calibration after 13 months of storage.

FIGS. 8A-8B show collected lactate sensor data after cyanide injection in a rabbit, with continuous monitoring of lactate (FIG. 8A) and changes in lactate (FIG. 8B).

FIG. 8C shows continuous lactate monitoring during a clinical exercise study on a human.

FIGS. 9A-9B show confocal microscopy images of porous PTFE 10 μm+dye.

FIGS. 9C-9D show confocal microscopy images of porous PTFE 10 μm+dye+PEGDM.

FIGS. 9E-9F show confocal microscopy images of porous PTFE 60 μm+dye+PEGDM.

FIG. 10A is a scanning electron microscopy (SEM) image of porous PTFE 10 μm+dye.

FIG. 10B shows an SEM image of porous PTFE 10 μm+dye+PEGDM.

FIG. 10C shows an SEM image of porous PTFE 10 μm+dye+PEGMM.

FIG. 11A shows an SEM image of porous PTFE 60 μm+dye.

FIG. 11B shows an SEM image of porous PTFE 60 μm+dye+PEGDM.

FIG. 11C shows an SEM image of porous ePTFE hydrophilic 10 μm+dye.

FIG. 11D shows an SEM image of porous ePTFE hydrophilic 10 μm+dye+PEGDM.

### DETAILED DESCRIPTION OF THE INVENTION

As will be described herein, the present invention features a composite matrix comprising a porous mesh sheet (solid phase or scaffold), in which at least some portion of the mesh (or walls of the pores) are coated with a luminescent material and at least one polymer mixture that contains target detecting molecules and entrapped within the pores of the mesh sheet. The polymer mixture is also porous, with pores having typically having characteristic size on the nanometer scale. These nanometer scale pores provide selective passages to analytes of choice. Additionally, the polymer mixture pores are orders of magnitude smaller than cells and thus prevent cellular or tissue infiltration when the composite matrix is implanted, thus allowing for ease of removal. The mesh pores have characteristic size of a few microns or smaller, further preventing tissue and cell infiltration. Further still, due to the designed configuration of the composite matrix constituents, the present invention achieved higher detection efficiencies, higher signal intensities, and smaller necessary dimensions, which as a result lead to faster diffusional dynamics and response time to changes. Such fast dynamics better represent the concentration changes of analytes in the interstitial fluid and within the body in real-time, and therefore, the need to infiltrate tissue and capillaries into the implant in order to provide close proximity between the embedded target detecting molecules and blood vessels becomes unnecessary.

### I. Composite Matrix

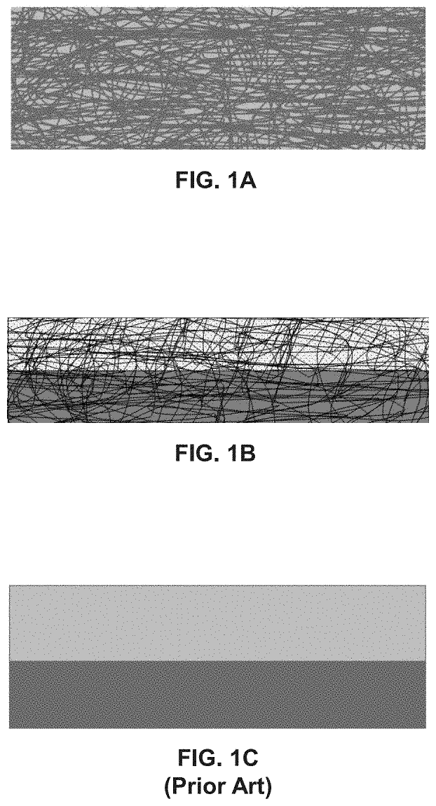

According to some embodiments, the present invention features a composite matrix for analyte biosensors. Referring to FIG. 1A, the composite matrix may comprise a porous mesh, a dye that coats the porous mesh, and an enzyme mixture entrapped in the pores of the dye-coated mesh. As an example, in some embodiments, the composite matrix comprises a porous polytetrafluoroethylene (PTFE) sheet, a polystyrene-dye composite, and a homogenous mixture made from a photo-curable hydrogel, enzyme molecules, and photoinitiators. FIG. 1A shows the composite geometry and the entrapment of enzyme mixture inside the dye coated porous mesh. This geometry provides high surface areas and therefore high luminescence. Additionally, closer proximity of the dye particles to the enzyme molecules can result in increased sensitivity.

According to other embodiments, the composite matrix may comprise a porous mesh, a luminescent material disposed in the porous mesh, and at least one target-detecting ixture entrapped in the pores of the mesh. As an example, the composite matrix may comprise a porous polytetrafluoroethylene (PTFE) sheet, a polystyrene-dye composite, and a homogenous mixture made from a photo-curable hydrogel, enzyme molecules, and photoinitiators. In another embodiment, the composite matrix may comprise a porous PTFE sheet, reversible binding probes modulating luminescence using FOrster Resonant Energy Transfer, Bioluminescence Resonant Energy Transfer, or quenching, where these probes are trapped in the pores of the mesh and target-selective, and a hydrogel mixture.

According to other embodiments, the composite matrix may comprise a porous mesh sheet, and one or more polymer mixtures entrapped in the porous mesh sheet, where at least one of the polymer mixtures comprises target-detecting molecules. In some embodiments, the composite matrix may further comprise a luminescent material.

In one embodiment, the mesh sheet may comprise a polymeric network of fibers and pores or gaps between the fibers. The luminescent material coats at least a portion of polymer fibers or pore walls that make up the mesh sheet. The luminescent material may also be disposed between the polymer fibers or pore walls. The polymer mixtures fill at least a portion of the pores or gaps between the fiber. The target-detecting molecules can release or produce or consume a substance after interaction with a stimulus, which causes the luminescent material to luminesce.

Referring to FIG. 1B, in some embodiments, the composite matrix contains different hydrogels on opposing sides. Different types of hydrogels with different molecular weights or characteristics are infused into opposite sides of the porous mesh to control the diffusion dynamics, filter entrance of interfering or detrimental molecules, or enclose the targeting molecules into large water filled pores of a bottom layer hydrogel. In this configuration, the composite can be placed in a frame to limit the diffusional pathway to only the top surface. With different molecular weights of the hydrogel, the top surface can act as a filter to unnecessary proteins or enzymes, or drug molecules which can interfere with the function or affect the binding proteins. In addition, it can act as a diffusion limiting factor or a cap that traps the targeting molecules placed within the lower level (hydrogel) and prevents them from leaching. With this configuration, the pore sizes of the lower layer hydrogel can be large for certain targeting molecules that require freedom of space. Moreover, this double layer configuration can be used to create a double sensor within one film. For example, the top surface can contain target detecting molecules that result in changes of emission dynamics, which can be recorded and correlated to an analyte level, while the lower layer can contain a conductive hydrogel with additives and necessary target detecting molecules that result in an electron exchange correlating to the level of a second analyte, this can be detected through electrochemical reactions and electrodes.

In some embodiments, the composite matrix can be electrically conductive. Such material can be used in optical or electrode-based electrochemical sensing of analytes, or in both methods simultaneously. For example, when two different reactions with two different analytes occur within one composite film, where one set of reaction results in electron release and the other does not, and the electron release only affects other properties of the composite material (spectral characteristics, volume change, etc.), both reactions can be recorded through simultaneous electrochemical and optical sensing.

### I-A. Porous Mesh

In some embodiments, the porous mesh may comprise a hydrophobic material. In one embodiment, the porous mesh comprises PTFE. However, the present invention is not limited to PTFE, and any appropriate biocompatible porous mesh may be used. In some embodiment, the porous mesh can be made from polymers. In one embodiment, this polymer can be expanded, etched or electrospun PTFE. In other embodiments, any porous hydrophobic polymer or ceramic or metallic membrane mesh can be used. Non-limiting examples of such materials are silver, alumina oxide, cellulose acetate, ceramics, sol gels, glass fiber filets, mixed cellulose ester, nylon, ethylene tetrafluoroethylene (ETFE), fluorinated ethylenepropylene (FEP), perfluoroalkoxy (PFA), polyacrylonitrile (PAN), polycarbonate (POTS), polyether ether ketone (PEEK), polyethersulfone (PES), polyester, polypropylene, and polyvinylidene fluoride (PVDF). In other embodiments, the porous mesh sheet may be fabricated from material made porous through methods such as 3D printing, laser cutting, salt leaching, connecting fibers, etching etc. to provide a porous structure. Such materials can be made of synthetic polymers, naturally occurring polymers, or combinations thereof. Non-limiting examples of such polymers include: 2-hydroxy methacrylate (HEMA), polyethylene glycol (PEG), polyvinyl alcohol, polyacrylamide and their mixtures, silicone, polyurethane, alginate, complex carbohydrates, extracellular matrix, fibronectin, chitosans, albumin, collagens, etc. In other embodiments, the porous mesh sheet may be fabricated from non-polymer materials such as ceramics or metals.

In some embodiments, the porous sheet or mesh has a thickness as low as 1 μm or less. The thickness of the porous sheet or mesh may be 1,000 μm or more. In other embodiments, the thickness of the porous sheet or mesh may range from 10 μm-50 μm, 50 μm-100 μm, 100 μm-200 μm, 200 μm-500 μm, 500 μm-800 μm, 800 μm-1,000 μm, or any range above, below or in between. The thickness of the porous sheet or mesh is not limited to these dimensions. The thickness of the porous sheet or mesh may be determined based on its application. In some embodiments, the porous sheet or mesh may be expanded PTFE (ePTFE), which is a PTFE sheet that has been stretched out to impart pores in the sheet.

### I-B. Luminescent Material

As used herein, the term “luminescent material” refers to compounds and molecular reporters that emit light.

According to one embodiment, the luminescent material may comprise a dye composite (e.g., polystyrene dye composite). The polystyrene can entrap the dye, or in some embodiments, dye particles. The dye composite coats the pores of the porous mesh or sheet (e.g., ePTFE) and/or the fibers of the porous mesh or sheet (e.g., PTFE sheet) and/or the pore walls, and may be further disposed between the polymer fibers or pore walls. In some embodiments, other matrices can be used for entrapment of dye particles. Non-limiting examples thereof include polyethylene glycol, poly (styrene-co-acrylonitrile), fluoroacrylic, sol-gel, poly(norobornene)s, polyvinylchloride, hydrophilic poly(2-hydroxyethyl methacrylate)-co-polyacrylamide, hydrophobic polystyrene, silicon rubbers, organically modified silica, ethyl cellulose, PVC, polysulfones, and poly(dimethylsiloxane).

Without wishing to limit the present invention, the porous mesh or sheet (e.g., PTFE sheet) provides an increase in surface area to which the dye is coated, thereby increasing the luminescence of the composite matrix. In preferred embodiments, the dye may be a luminescent oxygen sensitive dye. In other embodiments, the luminescent dye can be used to measure pH, carbon dioxide, partial pressure, sodium, potassium, magnesium, thiol, and/or calcium levels. Non-limiting examples of said dyes include porphyrin dyes, such as Pt(II) meso tetraphenyl tetrabenzoporphoryn (PtTPTBP) and other metalloporphyrins such as PtOEP, PdOEP, PtTFPP, PdTFPP, PtOEPK, PdOEPK, PtIFPPL, PdTFPPL, PdTPTBP, PtTPTBPF, PdTPTBPF, Pt1NF, Pd1NF, Pt2NF, Pd2NF, Pt3NF, Pd3NF, PtTPTNP, PdTPTNP, PtTBP(CO₂Bu)₈, PdTBP(CO₂Bu)₈, PtNTBP, PdNTBP, Oxyphor R2, Oxyphor G2, PtTCPP. Non-limiting examples of pH dyes include fluorescein and fluorescein derivatives, carboxyfluorescein, fluorescein sulfonic acid and its diacetate, carboxyphathofluorescein, 8-hydroxypyrene-1,3,6-trisulfonic acid (HPTS), nigercin, 5-(and-6)-carboxyfluorescein, 7-hydroxycoumarin-3-carboxylic acid and rhodamine B octadecyl ester perchlorate. Additional dyes include, but are not limited to, cyclometallated complexes such as Ir(III) or Pt(II): Ir(ppy)₃, Ir(ppy-NPh₂)₃, Ir(btpy)₃, and transition metals polypyridyl complexes such as [Ru(bpy)₃]²⁺ and [Ru(dpp)₃]²⁺.

According to another embodiment, the luminescent material may include reversible binding probes modulating luminescence using effects such as FOrster Resonant Energy Transfer (FRET), Bioluminescence Resonant Energy Transfer, or quenching. An example of said probes is luminescent lanthanide cryptates.

### I-C. Enzyme Mixture

In some embodiments, the enzyme-polymer mixture comprises a polymer and enzyme molecules entrapped in the polymer. The homogenous enzyme mixture is entrapped in the pores of the dye-coated mesh. The enzyme molecules are non-covalently entrapped within or covalently bound to the polymer. For example, the polystyrene-dye coated PTFE is loaded with the polymer-enzyme matrix to entrap the enzyme in between the chains of the polymer. In some embodiments, the polymer can be hydrophilic hydrogels. In other embodiments, the polymer mixtures comprise different types of hydrogels having similar or different molecular weight cutoffs. The hydrogels are configured to control diffusion and permeability, limit entrance into the other layers, or prevent leaching. In some embodiments, the hydrogel can be polymerized using photopolymerization and may further include a photoinitiator. In other embodiments, the hydrogels can be polymerized using different methods including bulk polymerization, solution polymerization, suspension or inverse suspension polymerization, grafting onto a support substrate, ionic crosslinking and temperature.

In some embodiments, the photocurable hydrogels may be polyethylene glycol (PEG) and/or its derivatives. Non-limiting examples of hydrogels that can be used in this application include: polyethylene glycol (PEG) and its derivatives, including but not limited to, poly(ethylene oxide), poly(3,4-ethylenedioxythiophene), 4arm-PEG, bis-PEG lauryl terminated, PEG monodisperse solution, catalase-polyethylene glycol, O,O′-Bis[2-(N-Succinimidyl-succinylamino)ethyl] PEG, PEG methyl ether methacrylate, diethylene glycol butyl ether methacrylate, PEG methacrylate, poly [silicone-a/t-PEG] dimethacrylate, PEG dimethacrylate, tetraethylene glycol dimethacrylate, tetra(ethylene glycol) diacrylate, Tri(ethyleneglycol) diacrylate, PEG diacrylate, PEG-polyisobutylene, PEG-poly(c-benzyl L-glutamate), and PEG-PNIPAAm.

In other embodiments, the hydrogel precursor can be collagen, fibrin, hyaluronic acid, alginate, agarose, dextran, poly(N-isopropylacrylamide) (PNIPAAm), poly(hydroxyethyl methacrylate) (PHEMA), poly(vinylpyrrolidone) (PVP), pullulan, Poly-lactic-co-glycolic acid(PLGA), poly(butylene terephthalate), poly(2-ethyl-2-oxazoline)-PCL, PLLA, PDLA, poly(vinyl alcohol) (PVA), chitosan-PVA, gelatin, albumin, polysaccharides, polyesters, polyamides, poly(oligoethylene glycol methacrylate) (POEGMA), phosphorylcholine, poly(vinylpyrrolidinone), poly(N-isopropylacrylamide) (PNIPAM), polyacrylamide (PAM), poly(-oxazoline), polyethylenimine (PEI), poly(acrylic acid), polymethacrylate, polyelectrolytes, chitosan-based hydrogels, chondroitin sulfate, hydroxypropylmethylcellulose, amylose, amylopectin, carrageenan, methylcellulose, hydroxypropylcellulose, poly(acrylamide), hydroxyethyl methacrylate (HEMA), 2-HEMA, poly(2-HEMA), poly-L-lysine, poly(propylene fumarate) cellulose derivatives, elastin, calcium polyphosphate, etc.

In some embodiments, one type of hydrogel or a combination of hydrogels may be used in the composite matrix. In other embodiments, one or multiple layers of hydrogel, each with different molecular weights may be used in the composite matrix. The top layer can filter unnecessary proteins or enzymes, or drug molecules which can interfere with the function or affect the binding proteins.

In some embodiments, particles or nanoparticles may also be incorporated into the hydrogel matrix to provide electronic conductivity, mechanical strength, luminescent properties, or magnetic properties. These particles may include but are not limited to gold nanoparticles, silver nanoparticles, etc. In other embodiments, the hydrogels themselves may be conductive, such as polypyrrole (PPy), polyaniline (PANi), polythiophene (PT), and poly(3,4-ethylene dioxythiophene) (PEDOT).

In some embodiments, the additives can be incorporated in the hydrogel matrix. In some embodiments, target detecting molecules can be labeled with an additional molecule. These additives are cofactors that may work in parallel with target detection molecules. In other embodiments, these additives are particles/nanoparticle. Such particles may be placed inside a stabilizing gel and mixed into the hydrogel matrix. In another embodiment, these particles are non-covalently immobilized into the hydrogel matrix. In yet another embodiment, these particles are covalently immobilized into the hydrogel matrix. These particles can be metal based, ceramic based, or polymer based. Examples of additive particles include, but are not limited to, gold nanoparticles, silver nanoparticles, platinum nanoparticles, iron oxide, zinc oxide, titanium dioxide, cadmium selenide (CdSe) or cadmium telluride (CdTe). Without wishing to be bound to a particular theory or mechanism, incorporation of the particles can result in enhanced mechanical and spectral properties of the composite as well as magnetic characteristics or electronic conductivity of the composite film, which can be utilized in electrode-based electrochemical analyte sensing. In some embodiments, these additives can be used to report volume changes in the hydrogel matrix. In such cases, volume change of the hydrogels (swelling/shrinking) can result in changes to the optical response of the composite sheet. Such changes compared to a reference can correlate to changes in hydration of the subject.

Other non-limiting examples of the additives include carbon nanotubes, quantum dots, and FRET molecules such as luminescent lanthanide cryptates. For example, changes in volume within the hydrogel infused composite matrix that includes gold nanoparticles as additives can result in changes in optical diffraction of the matrix, and therefore a change in a detectable signal that can correlate to hydration. As another example, the composite matrix can include a separate pH sensitive luminescence molecule as an additive, which can report pH changes within the matrix caused by changes in emission peak of a luminance which is chosen to be spectrally distinct from the analyte sensing or oxygen sensing dye component. In such embodiments, the sensing characteristics of the composite matrix can be altered or advanced to generate signals that can detect different and additional analytes or stimuli. Such additives can result in detectable signals that would indicate changes in response to a stimulus. Changes within the matrix that are recordable through introducing such additives are, for example, changes in volume (swelling/shrinking), pressure, temperature, optical diffraction, absorbance FRET or quenching, ultrasonic, acoustic, or radiation signals, as well as other measurable responses to a stimulus. Moreover, the composite matrix may be used to measure levels of analytes in small blood drops.

In one embodiment, the enzyme is not covalently attached to the hydrogel, but instead is entrapped in pores so as to not interfere with enzyme conformational changes to maintain high activity. In an alternative embodiment, the enzyme may be covalently attached to the hydrogel. Following UV light polymerization of the composite, a robust sheet with high mechanical stability is formed. Without wishing to limit the present invention, the dye-coated mesh can act as a scaffold that absorbs the hydrogel-enzyme mixture and it can remarkably prevent the mixture from falling out of the composite sheet.

Examples of the enzyme molecules include, but are not limited to, glucose oxidase and lactate oxidase, lactate oxidative decarboxylase, lactic oxygenase, lactate oxygenase, lactic oxidase, L-lactate monooxygenase, lactate monooxygenase, L-lactate-2-monooxygenase, cholesterol oxidase, alcohol oxidase, bilirubin oxidase, ascorbate oxidase, choline oxidase, pyruvate oxidase, sarcosine oxidase, tyramine oxidase, Acyl-CoA oxidase, NADPH oxidase, cortisol enzymes, and the combination of R(3) hydroxybutanoate and NAD+.

In other embodiments, the enzyme may belong to the family of oxidoreductases. In this case, the matrix may also contain cofactors including, but not limited to, NADP and NAD+. In such embodiments, the matrix can also include other proteins or antibodies. In this embodiment, the electron transfer within a conductive hydrogel matrix can be recorded and correlated to the level of analytes in terms of electrochemical sensors. In another approach, the changes in H+ due to the chemical reactions can affect the emission dynamics a pH sensitive embedded luminescent dye. Examples of such enzyme molecules include but are not limited to: alcohol dehydrogenase, glycerol dehydrogenase, galacticol 2-dehydrogenase, aldehyde reductase, L-lactate dehydrogenase, D-lactate dehydrogenase, glycerate dehydrogenase, 3-hydroxyglutanoate dehydrogenase, 3-hydroxyisobutyrate dehydrogenase, glucose 1-dehydrogenase, D-galactose 1-dehydrogenase, 3-hydroxypropionate dehydrogenase, and 3-alpha-hydroxysteroid dehydrogenase.

In some non-limiting embodiments, the photoinitiator is 2,2-dimethoxy-2-phenyacetophenone. In other non-limiting embodiments, the photointiators may be acetophenone, anisoin, anthraquinone, anthraquinone-2-sulfonic acid, sodium salt monohydrate, (benzene) tricarbonylchromium, benzil, benzoin, benzoin ethyl ether, benzoin isobutyl ether, benzoin methyl ether, benzophenone, benzophenone/1-hydroxycyclohexyl phenyl ketone, 3,3′,4,4′-benzophenone-tetracarboxylic dianhydride, 4-benzoylbiphenyl, 2-benzyl-2-(dimethylamino)-4′-morpholinobutyrophenone, 4,4′-bis(diethylamino)benzophenone, 4,4′-bis(dimethylamino)benzophenone, camphor quinone-2-chlorothioxanthen-9-one, 2,2-diethoxyacetophenone, dibenzosuberenone, 4,4′-dihydroxybenzophenone, (cumene)cyclopentadienyliron(ii)hexafluorophosphate, 4-(dimethylamino)benzophenone, 4,4′-dimethylbenzil, 2,5-dimethylbenzophenone, 3,4-dimethylbenzophenone, diphenyl(2,4,6trimethylbenzoyl)-phosphine oxide/2-hydroxy-2-methylpropiophenone, 4′-ethoxyacetophenone, 2-ethylanthraquinone, ferrocene, 3′-hydroxyacetophenone, 4′-hydroxyacetophenone, 3-hydroxybenzophenone, 4-hydroxybenzophenone, 1-hydroxycyclohexyl phenyl ketone, 2-hydroxy-2-methylpropio-phenone, 2-methyl-benzophenone, 3-methylbenzophenone, methybenzoylformate, 2-methyl-4′-(methylthio)-2-morpholino-propiophenone, etc.

### II. Biosensors

In some embodiments, the sensor may comprise a light source and the composite matrix. The composite matrix is disposed within the light path of the light source. The sensor may further comprise a detector circuit capable of recording light emissions from the composite matrix. The light source may be a light-emitting diode (LED). The composite matrix is attached to the LED via attachment through an adhesive. In other embodiments, the sensor may further comprise electrodes, flexible circuits, a signal detector, a signal receiver, a signal computing processor, a signal transmitter, a power source, a data storage component, a data transmitter, or a combination thereof.

In some embodiments, the sensor may comprise an electrode and the composite matrix that is conductive. The composite matrix may be coupled to the electrode, for example, via an adhesive. The sensor may further comprise a detector circuit coupled to the electrode for detecting signals. Non-limiting examples of signals include voltage, impedance, current, electric field, magnetic field, ultrasonic, acoustic, or radiation. In other embodiments, the sensor may further comprise flexible circuits, a signal detector, a signal receiver, a signal computing processor, a signal transmitter, a power source, a data storage component, a data transmitter, or a combination thereof.

In one embodiment, the present invention also provides biosensors, e.g., oxidase-based biosensors used in continuous monitoring of an analyte produced or consumed or modified from an enzyme reaction. In this enzyme reaction, the enzymes consume oxygen upon catalyzing the biochemical reaction with its analyte. Exemplary enzyme reaction schemes are shown in Scheme 1 and 2 for Glucose Oxidase and Lactate Oxidase, respectively. This consumption of oxygen can be measured using the luminescence oxygen sensitive dyes in the composite sheet to determine dissolved oxygen concentration.

Scheme 1: Glucose Oxidase (GOX): glucose+O₂→gluconic acid (GA)+H₂O₂

Scheme 2: Lactate Oxidase (LOX): L-Lactate+O₂→pyruvate+H₂O₂

The oxidase enzyme-based biosensors may comprise the composite matrix sheet attached to a light source. In some embodiments, a biocompatible adhesive, such as ethyl cyanoacrylate, may be used to adhere the sheet to the light source. The adhesive can polymerize upon exposure to humidity resulting in a strong bond between the sheet and light source. In some embodiments, the light source may comprise light emitting diodes (LEDs) soldered onto a flex circuit. The present invention is not limited to the aforementioned adhesives or light sources.

Without wishing to limit the present invention, the mechanical stability of the luminescence dye/enzyme matrix is highly increased by using a porous mesh. This can provide more secured protection for the reagent and lower the possibility of leaching in the body. Moreover, the size of the composite sheet containing all the components necessary for analyte detection is minimized (e.g., to as low as 30 μm in thickness or thinner), thereby making the analyte sensor overall thinner and insertion into the subcutaneous space less painful. In addition, high activity of the enzymes is maintained by the methods of entrapment, as compared to the method of covalent attachment of enzyme molecules to the hydrogel. Coating the pores/fibers of porous mesh (e.g., PTFE) with dye, which is in direct contact with the hydrogel can decrease the dynamic delay in response to changing bulk analyte concentrations. Further still, the diffusion rate through the composite matrix structure is limited due to the porous mesh constituent, thereby increasing the analyte sensitivity range.

In some embodiments, the luminescence dye-enzyme matrix component attachment to the light source is strengthened using highly viscous biocompatible ethyl cyanoacrylate adhesive. This improved adhesion is critical to maintaining sensor integrity during insertion into the tissue. The storage and operational stability is increased by the two-step entrapment method to entrap enzyme in a hydrogel matrix and the hydrogel matrix in the porous PTFE respectively. This methodology provides simple reproducibility for manufacturing purposes, potentially lowering costs and time.

In other embodiments, different configurations of the composite matrix, light source and detector may be implemented herein. In one embodiment, the composite matrix is adhered to light source and implanted, while the detector is place above the skin. For example, the composite matrix is adhered to the light source and implanted with a flexible circuit connected to a detector outside and above skin. In another embodiment, the composite matrix is implanted and the light source and detector are outside and above skin. In yet another embodiment, the composite sheet may be adhered to the light source and implanted along with the detector.

In embodiments for electrochemical sensing, the composite sheet may be adhered to an electrode and implanted, whereas the detector is placed outside the skin. In another embodiment, the composite sheet may be adhered to the electrode and detector and implanted into the skin.

In an alternative embodiment featuring a double layer composite matrix with the bottom layer being conductive and the top layer being luminescent, the composite sheet may be adhered to an electrode and implanted whereas the light source and detector are outside the skin. In another alternative embodiment, the composite sheet may be adhered to an electrode and light source and implanted while the detector is outside the skin. These embodiments may be used for double sensor composites.

In further embodiments, an analyte is released, consumed or produced due to chemical reactions. The biosensors may be used to detect or monitor the anlaytes or targets such as small molecules, ions, lipids, amino acids, peptides, polypeptides, proteins, glycoproteins, saccharides, nucleic acids and fragments thereof, insulin, glucose, lactate, oxygen, glucagon, cholesterols, triglycerides, HDL, glutamine, lactose, sucrose, pyruvate, cytokines, chemokines, eicosanoids, glycated hemoglobin (A1C), leptins, troponin, small molecule drugs, myoglobin, dopamine, serotonin, sodium, magnesium, calcium, potassium ions, hormones, metabolites, glycerol, ammonia, ethanol, methanol, CO₂, ketone, cortisol, pH, temperature, or a combination thereof that is important in health care and prevention. In addition to monitoring of analytes that are produced inside the body, the biosensor can be used to detect and monitor levels of exogenous molecules of importance for patients, healthcare professional, and hospitals. Examples of such monitoring can include, but is not limited to, continuous monitoring of food additives, caffeine, alcohol, nicotine, lead, vitamins, and other substances. Another non-limiting example is continuous measurement of chemotherapeutic agents for cancer treatment. This can help and provide feedback on the concentration of the drug in the patient's body and guide the treatment.

In some embodiments, the biosensor may be part of an analyte sensing system comprised of the following non-limiting components: the composite matrix, LEDs and electrodes, flexible circuits, signal detector, signal transmitter and receiver, signal computing processer, battery or other power source, data storage component such as a memory, and data transmitter. The sensing system can process the data or transmit the data to other systems such as hand-held devices, tablet, smart phone, smart watch, laptop, or desktop computer. The data collected from such sensors can be analyzed using a mathematical algorithm to predict the upcoming changes in levels of analytes or correlated hormones. Moreover, the continuous biosensor system can work in conjunction with secondary systems to deliver therapeutics based on the measured levels of analytes. One example of a secondary system is an insulin pump system working in conjunction with the analyte sensor of the present invention to create a closed loop system for advanced glycemic control.

According to other embodiments, the biosensor is part of a biosensing system. The biosensing system may comprise a sensor comprising a composite matrix with a porous mesh and at least one target-detecting polymer mixture disposed in the porous mesh, where the at least one target-detecting polymer mixture comprises a luminescent material and a conductive material; an electrode coupled to the composite matrix; a light source; and a detector. The sensor is configured to be implanted into a subject, and the light source and detector are disposed external to and on the skin. The light source is configured to illuminate the luminescent layer of the composite matrix and the detector is configured to detect emitted light. The sensor is configured to detect analytes by electrochemical sensing and optical sensing.

In some other embodiments, the biosensing system may comprise a double-layer sensor comprising a composite matrix comprising a porous mesh and two target-detecting polymer mixtures, each polymer mixture forming a layer within the porous mesh, where one of the layers comprises a luminescent material and the other layer comprises a conductive material; an electrode attached to the conductive layer; a light source; and a detector. The sensor is configured to be implanted into a subject such that the luminescent layer is adjacent to a skin, and the light source and detector are disposed external to and on the skin. The light source is configured to illuminate the luminescent layer of the composite matrix and the detector is configured to detect emitted light. Thus, the sensor can detect two analytes by electrochemical sensing and optical sensing.

In some embodiments, the biosensing system may further comprise a second detector coupled to electrode for detecting electrode signals. In other embodiments, the biosensing system may further comprise a flexible circuit, a signal receiver, a signal computing processor, a signal transmitter, a power source, a data storage device, a data transmitter, or combinations thereof.

In other embodiments, the biosensors described herein may be used to monitor levels of an analyte in a subject. In one embodiment, the method may comprise implanting a sensor into the subject, illuminating the composite matrix using the light source, thereby causing the composite matrix to emit light, and detecting an intensity and/or dynamics of the emitted light. The intensity or dynamics of emitted light corresponds to the levels of the analyte. In another embodiment, the method may comprise implanting into the subject a sensor, and detecting from the sensor a signal using the electrode, which corresponds to the levels of the analyte.

In yet other embodiments, the biosensors described herein may be used to monitor tissue hydration in a subject. In one embodiment, the method may comprise implanting a sensor into the subject, illuminating the composite matrix using the light source, thereby causing the composite matrix to emit light, and detecting an intensity and/or dynamics of the emitted light. The intensity or dynamics of emitted light corresponds to tissue water content. In another embodiment, the method may comprise implanting into the subject a sensor, and detecting from the sensor a signal using the electrode, which corresponds to the levels of the tissue water content.

### III. Methods of Producing Composite Matrix and Biosensors

According to some embodiments, the method of producing a composite matrix may comprise introducing a luminescent material into a porous mesh sheet, wherein the luminescent material coats at least a portion of the porous mesh sheet; infusing the porous mesh sheet with one or more target-detecting polymer mixtures, wherein the one or more target-detecting polymer mixtures becomes entrapped in the porous mesh sheet; and curing the one or more target-detecting polymer mixtures, thereby producing the composite matrix. The method may further comprise plasma-treating a surface of the porous mesh sheet with the luminescent material, thereby increasing hydrophilicity of the porous mesh sheet with the luminescent material. In other embodiments, the method may further comprise compressing the porous mesh sheet, thereby increasing penetration of the luminescent material and/or the one or more target-detecting polymer mixtures into the porous mesh sheet.

According to some embodiments, the method of producing a composite matrix may comprise infusing a porous mesh sheet with one or more target-detecting polymer mixtures so as to trap the one or more target-detecting polymer mixtures in the porous mesh sheet; and curing the one or more target-detecting polymer mixtures, thereby forming the composite matrix. The method may further comprise introducing a luminescent material into the porous mesh sheet prior to infusion of the polymer mixture into the porous mesh, such that the luminescent material coats at least a portion of the porous mesh sheet. In other embodiments, the method further comprises compressing the porous mesh sheet such that the one or more polymer mixtures penetrate the porous mesh sheet.

In some embodiments, two or more target-detecting polymer mixtures are infused into the mesh sheet, wherein each polymer mixture forms a layer within the mesh sheet. The target-detecting polymer mixtures may comprise at least two different types of hydrogel having similar or different molecular weight cutoffs. The target-detecting polymer mixtures further comprise particles or nanoparticles, cofactors, enzymes, or combinations thereof.

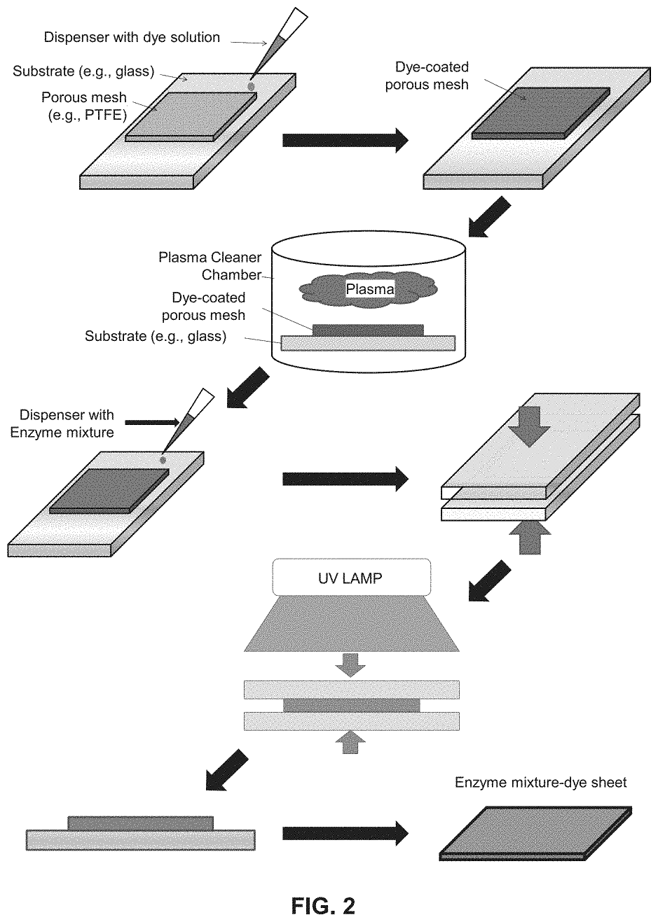

A non-limiting example of the production of a composite matrix is shown in FIG. 2. The present invention is not limited to the steps or the materials used in FIG. 2. Briefly, a photosensitive dye is applied to a porous mesh. The dye-coated porous mesh is allowed to fully evaporate out the solvent in which the dye was dissolved. Next, the dye-coated porous mesh is plasma treated to increase the surface hydrophilicity. Then, an enzyme mixture (e.g., PEG-enzyme mixture) is coated onto both sides of the sheet. The coated porous mesh with the impregnated enzyme-mixture is then placed in between two substrates (e.g., glass substrates) and pressure is applied to fully force the mixture to penetrate the coated sheet as well as limit the final thickness of the composite sheet. The substrates are then placed under UV lamp and removed from the composite sheet.

### EXAMPLES

The following are non-limiting examples of preparing the composite material of the present invention and use thereof in a biosensor. It is to be understood that the examples are for illustrative purposes only and are not intended to limit the present invention in any way. Equivalents or substitutes are within the scope of the invention.

### Fabrication Procedures

Enzyme molecules are homogenously mixed in photocurable PEG. A small amount of photoinitiator agent (2,2 dimethoxy-2-phenyacetophenone) is then added to the mixture to complete polymerization upon UV exposure. Relative masses are as follows: 1 ml PEG, 200 mg GOX, 20 mg photoinitiator. The mixture is stirred overnight at room temperature in a dark environment to achieve homogenous distribution of enzyme particles.

Referring to FIG. 2, a photosensitive dye (Pt(II) meso tetraphenyl tetrabenzoporphoryn) is used to coat a porous mesh Teflon membrane (PTFE). The porosity in the membranes was introduced by the supplier's expansion, or electrospinning fibers of PTFE (ePTFE, or expanded PTFE membrane). Other methods of creating pores in the PTFE may be used. The thickness of the porous PTFE membrane can be as low as 10 μm, however the present invention is not limited to 10 μm. Three manufactured PTFE sheet types, electrospun PTFE of thicknesses 10 μm and 60 μm sheet and hydrophilic ePTFE were used. The photosensitive dye is mixed with polystyrene and the mixture is then dissolved in chloroform (ratio: 1 mg dye to 15 mg polystyrene to 225 μl chloroform.) The sheet is then coated with the dye on both sides with the dye-containing solution. This coating can be done through different processes including but not limited to dye coating, spraying, convection, the like, etc. The dye coated sheets are then placed in a dark environment overnight to fully evaporate out the chloroform.

Next, the polystyrene-dye-PTFE sheet is then plasma treated on both sides each for a duration of approximately 5 minutes or less to increase the surface hydrophilicity. The polymerization time can changes based on the type of photoinititor or hydrogel used, the thickness of the film, the power of the UV lamp and so on. The PEG-enzyme mixture may then be then knife-coated onto both sides of the sheet. As shown in FIG. 2, the coated porous sheet with the impregnated enzyme-PEG mixture is then placed in between two substrates (e.g., glass substrates) and pressure is applied to fully force the mixture to penetrate the coated sheet as well as limit the final thickness of the composite sheet. The substrates are then placed under UV lamp for duration of 5 minutes on each side facing the UV light. Finally, the substrates are then removed and separated from the composite sheet. An oxygen reference sheet without enzyme or hydrogel was also fabricated, herein referred to as a reference or REF1. Analyte levels in the matrix are best determined when comparing to a tissue reference oxygen level in the surrounding environment. This is done through comparison of oxygen levels at the analyte sensor film with REF1.

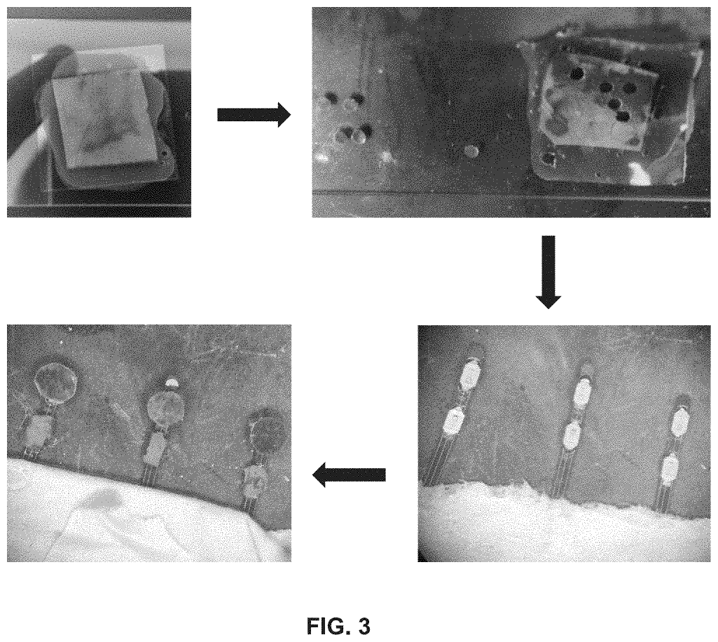

Referring to FIG. 3, the two sheets (with and without enzyme hydrogel) were then cut to small pieces and attached to light sources. In the biosensor device, small LEDs were soldered onto a flex circuit as the light source. The pieces of the sheets were attached to the light sources using highly viscous biocompatible ISO 10993 approved ethyl cyanoacrylate based Loctite® 4541, however, other biocompatible adhesives may be used. Additionally, other methods of placement including but not limited to geometrical and mechanical placement, surface chemistry attachment can also be used. The LEDs surfaces were knife coated with the adhesive and the cut sheets were pressed to adhere. The flex circuit was kept in a dark environment for 24 hours prior to first use to allow the adhesives to fully cure.

The methods may also include sterilization steps to sterilize the fabricated composite matrix. The sterilization technique can include, but is not limited to, ionizing radiation, such as gamma radiation, x-ray radiation and electron beam radiation. Additional methods of sterilization include ethylene oxide, ultraviolet light, and superheated steam.

### Results

The prepared flex circuits were connected to a developed sensor operated by National Instruments myRIO and LabVIEW software. The LEDs were turned on to excite the dye and quickly turned off. Immediately following the LED turning off, the emission decay from the oxygen sensitive dye was recorded by a photodiode for 200 μs at a sampling rate of 500 kHz. The data was analyzed in LabVIEW in real time. For each time point, 25 rapidly acquired decay measurements were averaged.

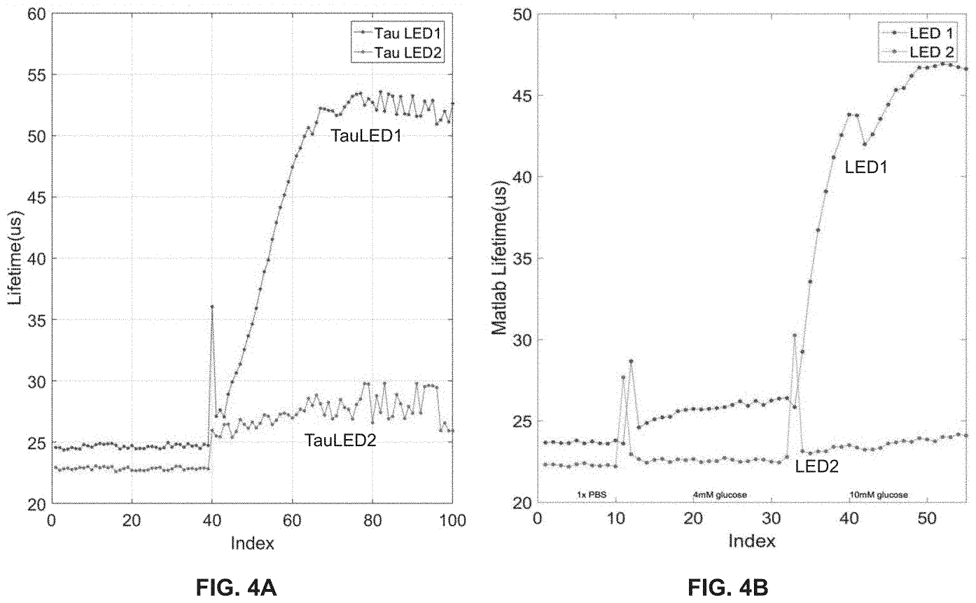

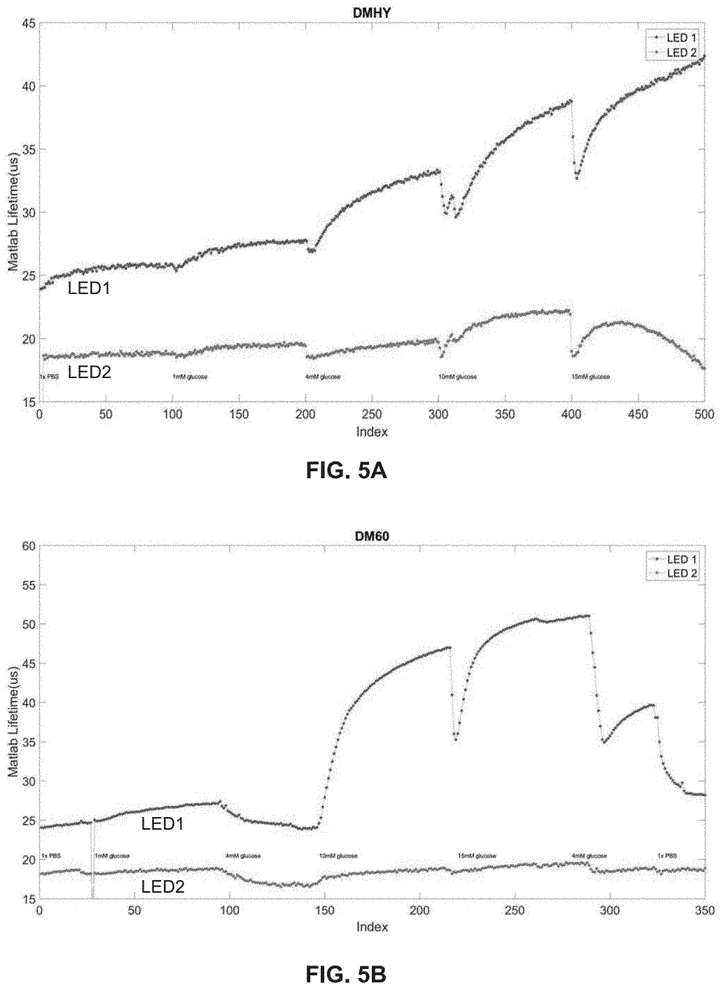

Referring to FIG. 4 and FIG. 5, the averaged data was used to calculate the decay lifetime values from both the enzyme-containing and the oxygen reference (no enzyme) sheets. The LEDs for each sheet were turned on in sequence so that the detector records light only from one sheet at a time. The analyte reacts with oxygen, thus depleting oxygen relative to the reference. Analyte concentration was computed by relating these two oxygen levels.

While decay was determined in the above calculation, the present invention is not limited to decay. In some other embodiments, other methods, such as phase shift, may be used in accordance with the present invention.

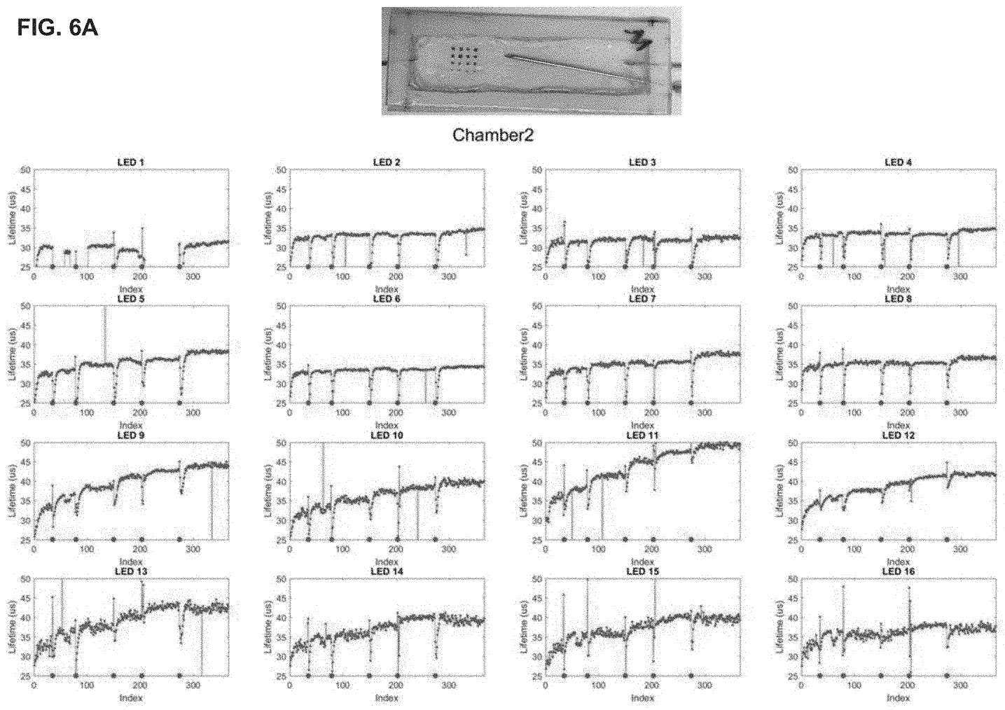

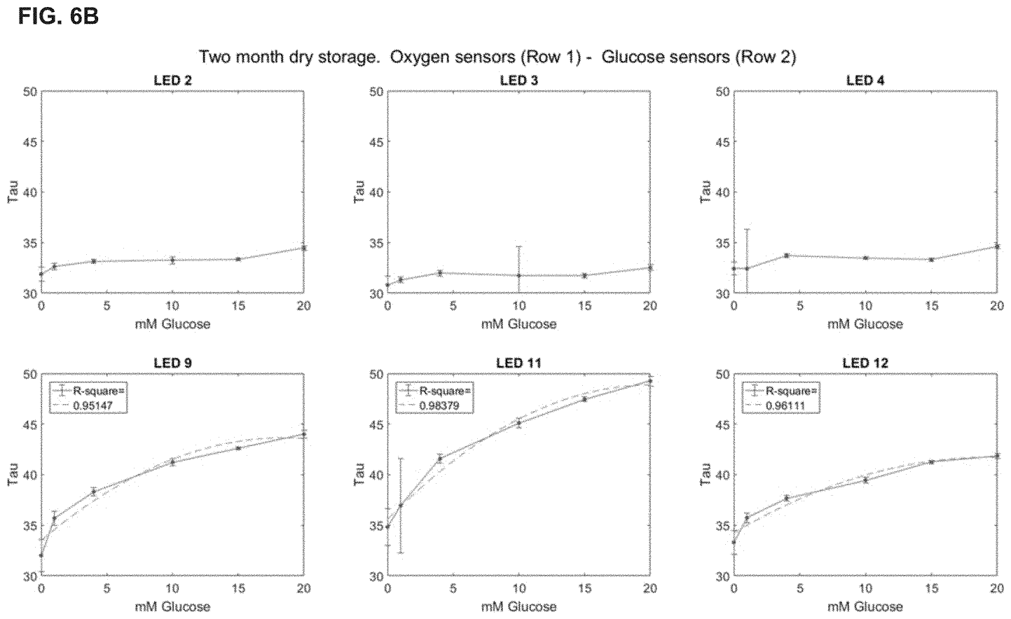

Referring to FIG. 6A and FIG. 6B, calibration of 4×4 array sensor chamber was done through inserting a series of solutions containing different analyte concentrations, for example ranging from pure 1×PBS to 1, 4, 10, 15, and 20 mM at room air or 5% oxygen. The lifetime decay constants were recorded for each condition. Lifetime constants for the reference films (row 1) remains stable over time and analyte concentration, whereas a step-wise increase in lifetime constant (caused by a decrease in oxygen quenching of the dye) was observed with increasing analyte concentration (rows 2:4). This stepwise behavior is indicative of sensor function. A calibration curve of the lifetime of the enzyme-sheet vs. analyte concentration has a functional form of polynomial order 2 with typical R²>0.95. This functional form was observed in all cases.

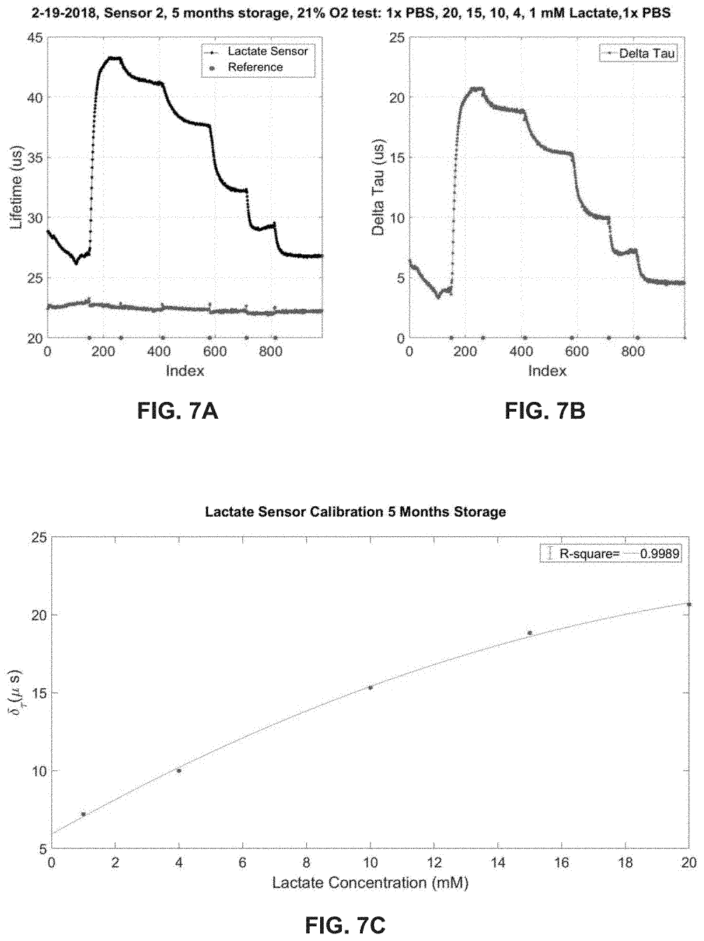

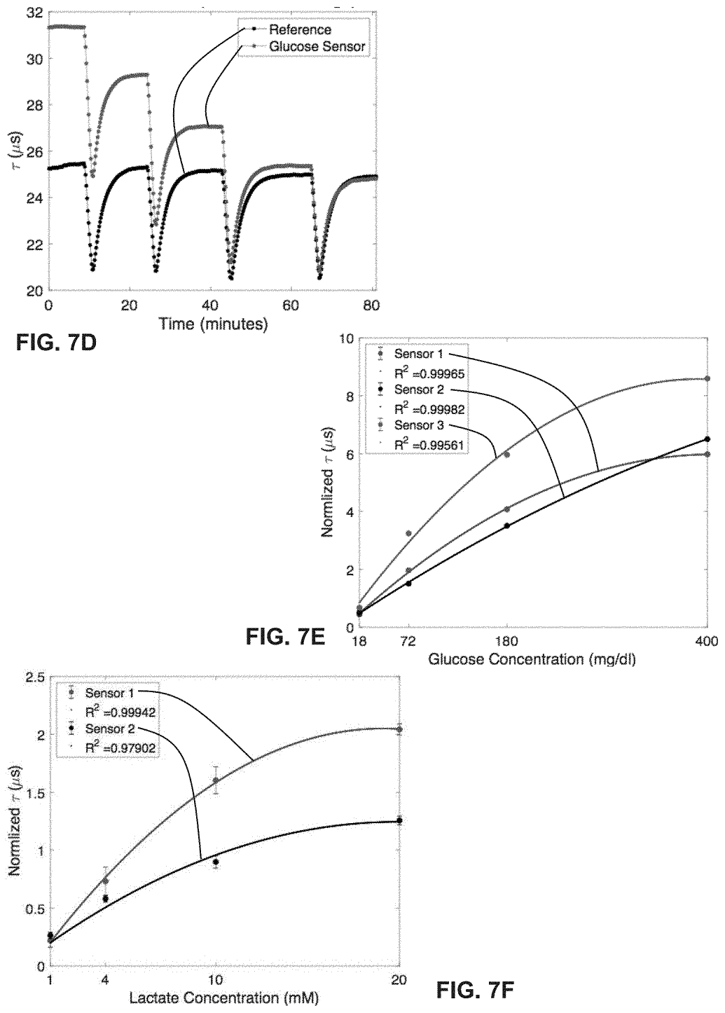

In FIGS. 7A-7C, the biosensors comprising the composite matrix maintain high levels of enzyme activity, which demonstrates that the present biosensors are capable of maintaining the activity and stability of enzymes overtime. The composite film used in this experiment was stored at room conditions for five months. The tip of the sensor is placed inside a fluid chamber and solutions containing different levels of lactate are then injected inside, the experiment is done at 21% oxygen. FIGS. 7A and 7B show that the reference film maintains its initial lifetime values throughout the experiment, confirming the continued existence of 21% oxygen in the fluid chamber. The enzymes in the analyte film of the sensor however undergo oxygen consumption after the injection of lactate solutions into the chamber. This oxygen consumption reduces the local oxygen levels and therefore increases the measured lifetime values of the analyte film. The measured lifetime values decrease with injection of solutions with lower levels of lactate. The difference between the lifetime values of the analyte film and the reference film (Delta Tau (δτ)) is then measured. Referring to FIG. 7C, the average of δτ at each lactate concentration is then used for finding a calibration curve between lifetime values and analyte level.

FIGS. 7D-7F show calibration of sensors made from composite sheets that were stored dry for 13 months. All calibrations were performed with continuous injection of 10% O₂ (76 mmHg) to simulate tissue oxygenation conditions. FIG. 7D shows calibration of a glucose sensor made from composite sheet at 400, 180, 72, 18 mg/dl glucose followed by 1× PBS. The step changes in τ in the glucose sensor sheet accompanied by the constant average T value reported from the reference oxygen sensor shows the functionality of the sensors. In FIGS. 7E and 7F, multiple glucose and lactate sensors made from composite sheets were calibrated. Analyzed results show a polynomial level 2 correlation between the Normalized τ and different analyte concentrations.

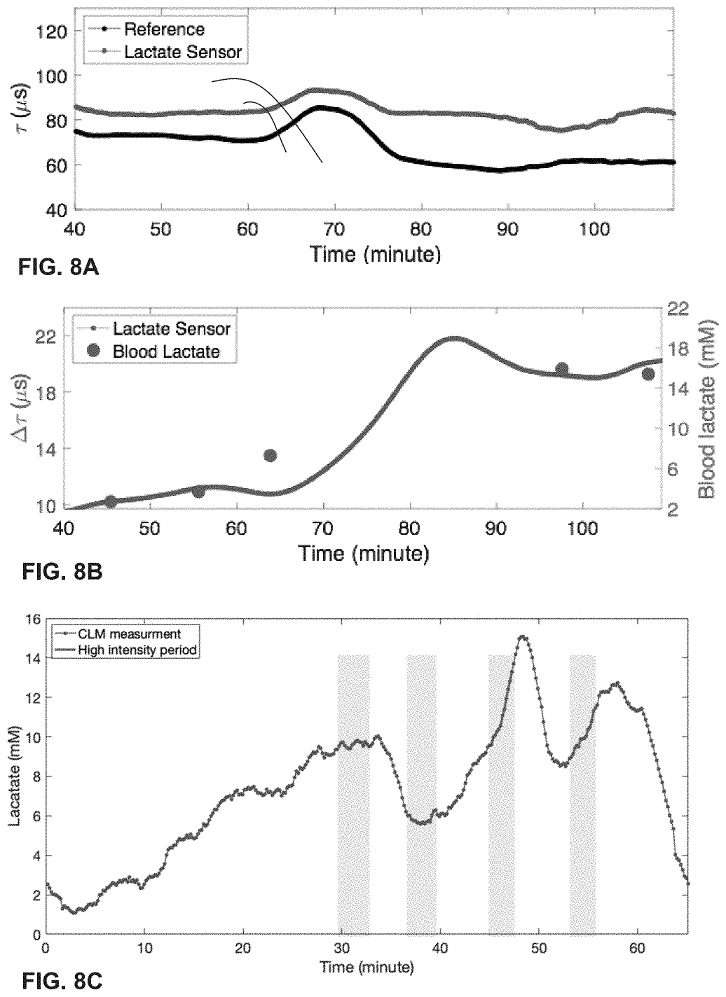

FIG. 8A-8B shows collected lactate sensor data from a pilot in vivo study of cyanide poisoning in a rabbit model to increase blood lactate levels. Cyanide injection starts 10 minutes and lasts for 30 minutes and the graphs show the continuous data collected after the cyanide injection stopped (40 minutes into the experiment). In FIG. 8A, lifetime values were collected from a subcutaneously inserted continuous lactate sensor. In FIG. 8B, an increasing trend in continuous calculated δτ may indicate the rise of lactate, which is also confirmed by reference blood lactate measurements (shown in solid circles). A lag time of around 6 minutes was observed.

FIG. 8C shows continuous lactate monitoring during a clinical exercise study performed on a 46 years old male subject with Type 1 diabetes. For this study, composite lactate films and oxygen reference films were adhered to surfaces of LEDs mounted on a flexible circuit. The tip of the flexible sensor was inserted underneath the skin, and the photodetector was placed above the skin in the path of the emission light and taped in place. The difference between the lifetime values of the analyte film and the reference film (δτ) was then measured and correlated with physiological levels of tissue lactate through post calibration, and graphed in this study. The subject undergoes a period of initial warm up, followed by four repeating sessions each containing periods of intense and moderate pedaling. The interval between each measurement is 22 seconds. The highlighted sections show the periods of intense pedaling. The results show that the increase and fall in tissue lactate levels correspond to the intense and moderate exercise periods respectively. Reference blood maximum values were estimated from historic highs of the participant during intense exercise. Moreover, results indicate that the lag time for the sensor was around 6 minutes. This study was performed with the help of exercise physiologists and physicians for safety with proper human study approvals.

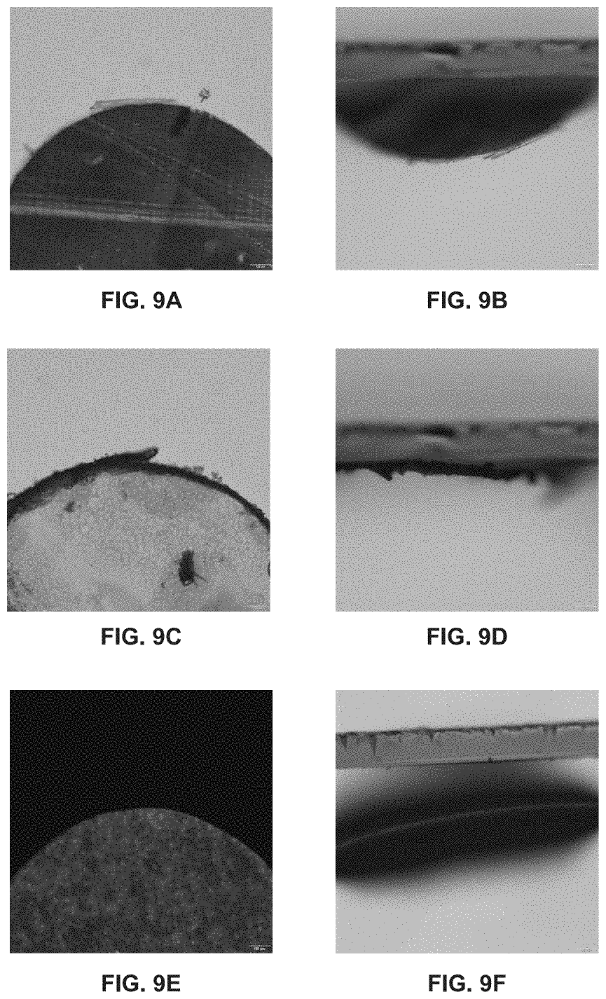

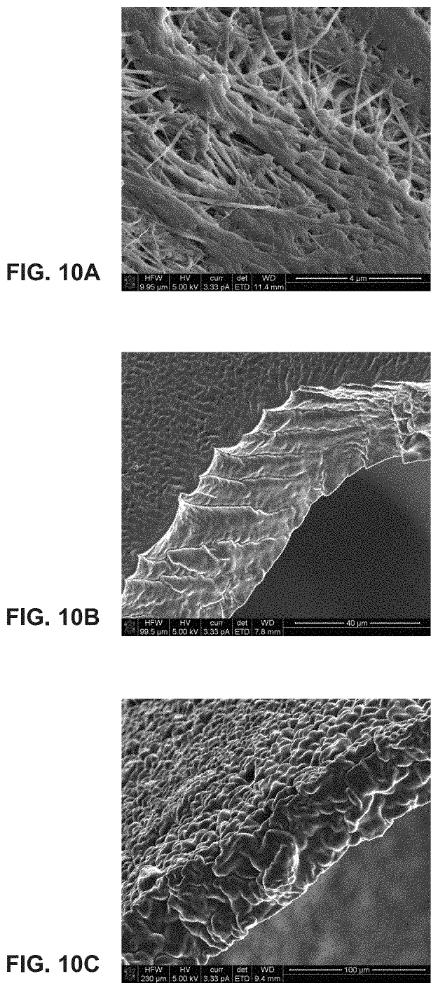

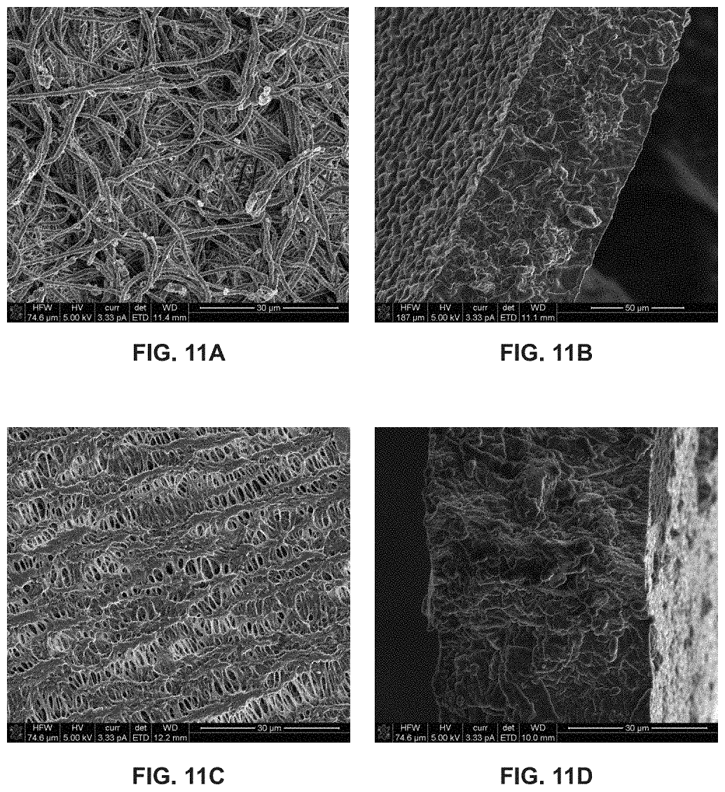

For illustrative purposes, FIGS. 9A and 9B show confocal microscopy images of porous PTFE 10 μm+dye. FIGS. 9C and 9D show confocal microscopy images of porous PTFE 10 μm+dye+PEGDM. FIGS. 9E and 9F show confocal microscopy images of porous PTFE 60 μm+dye+PEGDM. FIG. 10A shows a scanning electron microscopy (SEM) image of the composite matrix comprising porous PTFE 10 μm+dye. FIG. 10B shows an SEM image of the composite matrix comprising porous PTFE 10 μm+dye+PEGDM. FIG. 10C shows an SEM image of the composite matrix comprising porous PTFE 10 μm+dye+PEGMM. FIG. 11A shows an SEM image of the composite matrix comprising porous PTFE 60 μm+dye. FIG. 11B shows an SEM image of the composite matrix comprising porous PTFE 60 μm+dye+PEGDM. FIG. 11C shows an SEM image of the composite matrix comprising porous ePTFE hydrophilic 10 μm+dye. FIG. 11D shows an SEM image of the composite matrix comprising porous ePTFE hydrophilic 10 μm+dye+PEGDM.

The disclosures of the following U.S. Patents are incorporated in their entirety by reference herein: U.S. application Ser. No. 15/502,728. Various modifications of the invention, in addition to those described herein, will be apparent to those skilled in the art from the foregoing description. Such modifications are also intended to fall within the scope of the appended claims. Each reference cited in the present application is incorporated herein by reference in its entirety.

Although there has been shown and described the preferred embodiment of the present invention, it will be readily apparent to those skilled in the art that modifications may be made thereto which do not exceed the scope of the appended claims. Therefore, the scope of the invention is only to be limited by the following claims. Reference numbers recited in the claims are exemplary and solely for ease of examination of this patent application, and are not intended in any way to limit the scope of the claims to the particular features having the corresponding reference numbers in the drawings. In some embodiments, the figures presented in this patent application are drawn to scale, including the angles, ratios of dimensions, etc. In some embodiments, the figures are representative only and the claims are not limited by the dimensions of the figures. In some embodiments, descriptions of the inventions described herein using the phrase “comprising” includes embodiments that could be described as “consisting of”, and as such the written description requirement for claiming one or more embodiments of the present invention using the phrase “consisting of” is met.

What is claimed is:

1. A composite matrix for detecting a target, comprising:
a. a porous mesh material; and
b. at least one target-detecting polymer mixture filling the pores of the porous mesh material, said target-detecting polymer mixture comprising target-detecting molecules.

2. The composite matrix of claim 1, further comprising a luminescent material coating at least a portion of the porous mesh material.

3. The composite matrix of claim 2, wherein the target-detecting molecules interact with the target in such a way as to absorb or bind to or release or produce or consume a substance after interaction with a target, wherein said substance or target alters one or more luminescent properties of the luminescent material.

4. The composite matrix of claim 2, wherein the luminescent material coats at least a portion of polymer fibers or pore walls that make up the porous mesh material.

5. The composite matrix of claim 4, wherein the luminescent material is disposed between the polymer fibers or pore walls.

6. The composite matrix of claim 1, wherein the porous mesh material comprises a polymeric network of fibers and pores or gaps between the fibers.

7. The composite matrix of claim 6, wherein the at least one target-detecting polymer mixture fills at least a portion of the pores or gaps between the fibers.

8. The composite matrix of claim 1, wherein the at least one target-detecting polymer mixture further comprises particles or nanoparticles, cofactors, enzymes, or combinations thereof.

9. The composite matrix of claim 1, wherein the at least one target-detecting polymer mixture comprises two or more polymer mixtures, each polymer mixture of the two or more polymer mixtures forming a separate phase within the porous mesh material.

10. The composite matrix of claim 1, wherein the target-detecting molecules are non-covalently entrapped within at least one polymer or covalently bound to at least one polymer of the at least one target-detecting polymer mixture.

11. The composite matrix of claim 1, wherein the at least one target-detecting polymer mixture is a protein-polymer mixture that includes at least one enzyme.

12. The composite matrix of claim 11, wherein enzyme molecules of the protein-polymer mixture are non-covalently entrapped within or covalently bound to a polymer of the protein-polymer mixture.

13. The composite matrix of claim 1, wherein the at least one target-detecting polymer mixture comprise at least two different types of polymers having similar or different molecular weight cutoffs.

14. The composite matrix of claim 1, wherein a polymer of the at least one target-detecting polymer mixture is polymerized to entrap the target-detecting molecules in the polymer.

15. The composite matrix of claim 1, wherein the at least one target-detecting polymer mixture is electrically conductive.

16. A method of producing a composite matrix, said method comprising:
a. introducing a luminescent material into a porous mesh material, wherein the luminescent material coats at least a portion of the porous mesh material;
b. infusing the porous mesh material with one or more target-detecting polymer precursor mixtures, wherein the one or more target-detecting polymer precursor mixtures becomes entrapped in the porous mesh material; and
c. curing the one or more target-detecting polymer precursor mixtures, thereby producing the composite matrix.

17. The method of claim 16 further comprising applying pressure to the porous mesh material along with the luminescent material and/or the one or more target-detecting polymer precursor mixtures, thereby increasing penetration of the luminescent material and/or the one or more target-detecting polymer precursor mixtures into the porous mesh material.

18. The method of claim 17 further comprising plasma-treating the porous mesh material with the luminescent material, thereby increasing hydrophilicity of the porous mesh material with the luminescent material.

19. A method of producing a composite matrix, said method comprising:
a. infusing a porous mesh material with one or more target-detecting polymer precursor mixtures so as to fill the pores of the porous mesh material with the one or more target-detecting polymer precursor mixtures in the porous mesh material; and
b. curing the one or more target-detecting polymer precursor mixtures, thereby forming the composite matrix.

20. The method of claim 19, further comprising introducing a luminescent material into the porous mesh material prior to infusion of the polymer precursor mixture into the porous mesh, such that the luminescent material coats at least a portion of the porous mesh material.
